SEQUENCE LISTING (126 sequences)

SEQ ID NO: 1 - protein, 9 aa, Artificial Sequence
  synthetic peptide
EEYLKWATF

SEQ ID NO: 2 - protein, 9 aa, Artificial Sequence
  synthetic peptide
FWYLIMQVA

SEQ ID NO: 3 - protein, 12 aa, Artificial Sequence
  synthetic peptide
FWYLIMQVAHKR

SEQ ID NO: 4 - protein, 7 aa, Artificial Sequence
  synthetic peptide
FWYLIVM

SEQ ID NO: 5 - protein, 9 aa, Artificial Sequence
  synthetic peptide
DAQKLLEKM

SEQ ID NO: 6 - protein, 9 aa, Artificial Sequence
  synthetic peptide
DEELEQMLD

SEQ ID NO: 7 - protein, 9 aa, Artificial Sequence
  synthetic peptide
DQSLIYTLL

SEQ ID NO: 8 - protein, 9 aa, Artificial Sequence
  synthetic peptide
EELEADTEY

SEQ ID NO: 9 - protein, 9 aa, Artificial Sequence
  synthetic peptide
GEVAPSMFL

SEQ ID NO: 10 - protein, 9 aa, Artificial Sequence
  synthetic peptide
HEKVLNEAV

SEQ ID NO: 11 - protein, 9 aa, Artificial Sequence
  synthetic peptide
IAERYGFQY

SEQ ID NO: 12 - protein, 9 aa, Artificial Sequence
  synthetic peptide
KGPSDLLTV

SEQ ID NO: 13 - protein, 9 aa, Artificial Sequence
  synthetic peptide
LALTAPRPY

SEQ ID NO: 14 - protein, 9 aa, Artificial Sequence
  synthetic peptide
LEIDNRLCL

SEQ ID NO: 15 - protein, 9 aa, Artificial Sequence
  synthetic peptide
MEMFLFFTA

SEQ ID NO: 16 - protein, 9 aa, Artificial Sequence
  synthetic peptide
MEPGNNPIF

SEQ ID NO: 17 - protein, 9 aa, Artificial Sequence
  synthetic peptide
NGLEIIWAE

SEQ ID NO: 18 - protein, 9 aa, Artificial Sequence
  synthetic peptide
QEFWISQAS

SEQ ID NO: 19 - protein, 9 aa, Artificial Sequence
  synthetic peptide
QENEVLFTM

SEQ ID NO: 20 - protein, 9 aa, Artificial Sequence
  synthetic peptide
RERTMVSTR

SEQ ID NO: 21 - protein, 9 aa, Artificial Sequence
  synthetic peptide
RQFYLWTCL

SEQ ID NO: 22 - protein, 9 aa, Artificial Sequence
  synthetic peptide
SELLKEFPY

SEQ ID NO: 23 - protein, 9 aa, Artificial Sequence
  synthetic peptide
VENSFFLNV

SEQ ID NO: 24 - protein, 9 aa, Artificial Sequence
  synthetic peptide
YDGAYAPVL

SEQ ID NO: 25 - protein, 9 aa, Artificial Sequence
  synthetic peptide
DEQKLLEKM

SEQ ID NO: 26 - protein, 9 aa, Artificial Sequence
  synthetic peptide
DEELEQMLY

SEQ ID NO: 27 - protein, 9 aa, Artificial Sequence
  synthetic peptide
DESLIYTLL

SEQ ID NO: 28 - protein, 9 aa, Artificial Sequence
  synthetic peptide
EELEAHTEY

SEQ ID NO: 29 - protein, 9 aa, Artificial Sequence
  synthetic peptide
GEVAPRMFL

SEQ ID NO: 30 - protein, 9 aa, Artificial Sequence
  synthetic peptide
GEVAPDMFL

SEQ ID NO: 31 - protein, 9 aa, Artificial Sequence
  synthetic peptide
GEVAPEMFL

SEQ ID NO: 32 - protein, 9 aa, Artificial Sequence
  synthetic peptide
HEKVLNKAV

SEQ ID NO: 33 - protein, 9 aa, Artificial Sequence
  synthetic peptide
HEKVLNLAV

SEQ ID NO: 34 - protein, 9 aa, Artificial Sequence
  synthetic peptide
HEKVLNMAV

SEQ ID NO: 35 - protein, 9 aa, Artificial Sequence
  synthetic peptide
IEERYGFQY

SEQ ID NO: 36 - protein, 9 aa, Artificial Sequence
  synthetic peptide
KDPSDLLTV

SEQ ID NO: 37 - protein, 9 aa, Artificial Sequence
  synthetic peptide
KEPSDLLTV

SEQ ID NO: 38 - protein, 9 aa, Artificial Sequence
  synthetic peptide
KHPSDLLTV

SEQ ID NO: 39 - protein, 9 aa, Artificial Sequence
  synthetic peptide
KKPSDLLTV

SEQ ID NO: 40 - protein, 9 aa, Artificial Sequence
  synthetic peptide
KRPSDLLTV

SEQ ID NO: 41 - protein, 9 aa, Artificial Sequence
  synthetic peptide
LELTAPRPY

SEQ ID NO: 42 - protein, 9 aa, Artificial Sequence
  synthetic peptide
LEIDHRLCL

SEQ ID NO: 43 - protein, 9 aa, Artificial Sequence
  synthetic peptide
MEDFLFFTA

SEQ ID NO: 44 - protein, 9 aa, Artificial Sequence
  synthetic peptide
MEEFLFFTA

SEQ ID NO: 45 - protein, 9 aa, Artificial Sequence
  synthetic peptide
MERFLFFTA

SEQ ID NO: 46 - protein, 9 aa, Artificial Sequence
  synthetic peptide
MESGNNPIF

SEQ ID NO: 47 - protein, 9 aa, Artificial Sequence
  synthetic peptide
NELEIIWAE

SEQ ID NO: 48 - protein, 9 aa, Artificial Sequence
  synthetic peptide
QEFWISDAS

SEQ ID NO: 49 - protein, 9 aa, Artificial Sequence
  synthetic peptide
QEFWISEAS

SEQ ID NO: 50 - protein, 9 aa, Artificial Sequence
  synthetic peptide
QEFWISHAS

SEQ ID NO: 51 - protein, 9 aa, Artificial Sequence
  synthetic peptide
LENEVLFTM

SEQ ID NO: 52 - protein, 9 aa, Artificial Sequence
  synthetic peptide
RERDMVSTR

SEQ ID NO: 53 - protein, 9 aa, Artificial Sequence
  synthetic peptide
REREMVSTR

SEQ ID NO: 54 - protein, 9 aa, Artificial Sequence
  synthetic peptide
RERKMVSTR

SEQ ID NO: 55 - protein, 9 aa, Artificial Sequence
  synthetic peptide
RHFYLWTCL

SEQ ID NO: 56 - protein, 9 aa, Artificial Sequence
  synthetic peptide
SELLEEFPY

SEQ ID NO: 57 - protein, 9 aa, Artificial Sequence
  synthetic peptide
VENSFFLEV

SEQ ID NO: 58 - protein, 9 aa, Artificial Sequence
  synthetic peptide
YEGAYAPVL

SEQ ID NO: 59 - protein, 9 aa, Artificial Sequence
  synthetic peptide
CEECGKAFA

SEQ ID NO: 60 - protein, 8 aa, Artificial Sequence
  synthetic peptide
TEWPKRSL

SEQ ID NO: 61 - protein, 9 aa, Artificial Sequence
  synthetic peptide
EELAGNLWL

SEQ ID NO: 62 - protein, 9 aa, Artificial Sequence
  synthetic peptide
QESDSSFIL

SEQ ID NO: 63 - protein, 9 aa, Artificial Sequence
  synthetic peptide
MDMVCMSVF

SEQ ID NO: 64 - protein, 9 aa, Artificial Sequence
  synthetic peptide
NEFKFLKPI

SEQ ID NO: 65 - protein, 9 aa, Artificial Sequence
  synthetic peptide
TENVQFDHY

SEQ ID NO: 66 - protein, 9 aa, Artificial Sequence
  synthetic peptide
EELAGNLWL

SEQ ID NO: 67 - protein, 9 aa, Artificial Sequence
  synthetic peptide
QESDSSFIL

SEQ ID NO: 68 - protein, 9 aa, Artificial Sequence
  synthetic peptide
TENVQFDHY

SEQ ID NO: 69 - protein, 9 aa, Artificial Sequence
  synthetic peptide
DEQKLLEKM

SEQ ID NO: 70 - protein, 9 aa, Artificial Sequence
  synthetic peptide
IEPSRVVAI

SEQ ID NO: 71 - protein, 9 aa, Artificial Sequence
  synthetic peptide
EEFPYGIEA

SEQ ID NO: 72 - protein, 9 aa, Artificial Sequence
  synthetic peptide
EELMELLAA

SEQ ID NO: 73 - protein, 9 aa, Artificial Sequence
  synthetic peptide
IEERYGFQY

SEQ ID NO: 74 - protein, 9 aa, Artificial Sequence
  synthetic peptide
DESLIYTLL

SEQ ID NO: 75 - protein, 9 aa, Artificial Sequence
  synthetic peptide
QHSGLEKRF

SEQ ID NO: 76 - protein, 9 aa, Artificial Sequence
  synthetic peptide
VHSGLYPGL

SEQ ID NO: 77 - protein, 9 aa, Artificial Sequence
  synthetic peptide
AETQDSEIF

SEQ ID NO: 78 - protein, 9 aa, Artificial Sequence
  synthetic peptide
LELTAPRPY

SEQ ID NO: 79 - protein, 9 aa, Artificial Sequence
  synthetic peptide
PEALHTATF

SEQ ID NO: 80 - protein, 9 aa, Artificial Sequence
  synthetic peptide
IEGVPLSSY

SEQ ID NO: 81 - protein, 9 aa, Artificial Sequence
  synthetic peptide
WEALSVVLI

SEQ ID NO: 82 - protein, 9 aa, Artificial Sequence
  synthetic peptide
SELEGSTEI

SEQ ID NO: 83 - protein, 9 aa, Artificial Sequence
  synthetic peptide
LEFAVTFVL

SEQ ID NO: 84 - protein, 9 aa, Artificial Sequence
  synthetic peptide
AEPLCKYLL

SEQ ID NO: 85 - protein, 9 aa, Artificial Sequence
  synthetic peptide
REALRLPSF

SEQ ID NO: 86 - protein, 9 aa, Artificial Sequence
  synthetic peptide
KEPSDLLTV

SEQ ID NO: 87 - protein, 9 aa, Artificial Sequence
  synthetic peptide
LKLQIILFL

SEQ ID NO: 88 - protein, 9 aa, Artificial Sequence
  synthetic peptide
YRMGTSVER

SEQ ID NO: 89 - protein, 9 aa, Artificial Sequence
  synthetic peptide
NRGRCVAPY

SEQ ID NO: 90 - protein, 9 aa, Artificial Sequence
  synthetic peptide
METQIQQLL

SEQ ID NO: 91 - protein, 10 aa, Artificial Sequence
  synthetic peptide
GESPCPSEVL

SEQ ID NO: 92 - protein, 9 aa, Artificial Sequence
  synthetic peptide
CGECGKAFA

SEQ ID NO: 93 - protein, 9 aa, Artificial Sequence
  synthetic peptide
TKQWPKRSL

SEQ ID NO: 94 - protein, 9 aa, Artificial Sequence
  synthetic peptide
EGLAGNLWL

SEQ ID NO: 95 - protein, 9 aa, Artificial Sequence
  synthetic peptide
QKSDSSFIL

SEQ ID NO: 96 - protein, 9 aa, Artificial Sequence
  synthetic peptide
MNMVCMSVF

SEQ ID NO: 97 - protein, 9 aa, Artificial Sequence
  synthetic peptide
NKFKFLKPI

SEQ ID NO: 98 - protein, 9 aa, Artificial Sequence
  synthetic peptide
TGNVQFDHY

SEQ ID NO: 99 - protein, 9 aa, Artificial Sequence
  synthetic peptide
RGSDVIITL

SEQ ID NO: 100 - protein, 9 aa, Artificial Sequence
  synthetic peptide
IKPSRVVAI

SEQ ID NO: 101 - protein, 9 aa, Artificial Sequence
  synthetic peptide
IAERYGFQY

SEQ ID NO: 102 - protein, 9 aa, Artificial Sequence
  synthetic peptide
DAQKLLEKM

SEQ ID NO: 103 - protein, 9 aa, Artificial Sequence
  synthetic peptide
PKALHTATF

SEQ ID NO: 104 - protein, 9 aa, Artificial Sequence
  synthetic peptide
SQSELQVFW

SEQ ID NO: 105 - protein, 9 aa, Artificial Sequence
  synthetic peptide
SQLLLLACV

SEQ ID NO: 106 - protein, 9 aa, Artificial Sequence
  synthetic peptide
IQGVPLSSY

SEQ ID NO: 107 - protein, 9 aa, Artificial Sequence
  synthetic peptide
DQSLIYTLL

SEQ ID NO: 108 - protein, 9 aa, Artificial Sequence
  synthetic peptide
QLSGLEKRF

SEQ ID NO: 109 - protein, 9 aa, Artificial Sequence
  synthetic peptide
VDSGLYPGL

SEQ ID NO: 110 - protein, 9 aa, Artificial Sequence
  synthetic peptide
AGTQDSEIF

SEQ ID NO: 111 - protein, 9 aa, Artificial Sequence
  synthetic peptide
LALTAPRPY

SEQ ID NO: 112 - protein, 9 aa, Artificial Sequence
  synthetic peptide
PKALHTATF

SEQ ID NO: 113 - protein, 9 aa, Artificial Sequence
  synthetic peptide
IQGVPLSSY

SEQ ID NO: 114 - protein, 9 aa, Artificial Sequence
  synthetic peptide
WGALSVVLI

SEQ ID NO: 115 - protein, 9 aa, Artificial Sequence
  synthetic peptide
SQLEGSTEI

SEQ ID NO: 116 - protein, 9 aa, Artificial Sequence
  synthetic peptide
LVFAVTFVL

SEQ ID NO: 117 - protein, 9 aa, Artificial Sequence
  synthetic peptide
AGPLCKYLL

SEQ ID NO: 118 - protein, 9 aa, Artificial Sequence
  synthetic peptide
RGALRLPSF

SEQ ID NO: 119 - protein, 9 aa, Artificial Sequence
  synthetic peptide
KGPSDLLTV

SEQ ID NO: 120 - protein, 9 aa, Artificial Sequence
  synthetic peptide
LELQIILFL

SEQ ID NO: 121 - protein, 9 aa, Artificial Sequence
  synthetic peptide
YGMGTSVER

SEQ ID NO: 122 - protein, 9 aa, Artificial Sequence
  synthetic peptide
NGGRCVAPY

SEQ ID NO: 123 - protein, 9 aa, Artificial Sequence
  synthetic peptide
MATQIQQLL

SEQ ID NO: 124 - protein, 9 aa, Artificial Sequence
  synthetic peptide
GKSCPSEVL

SEQ ID NO: 125 - protein, 9 aa, Artificial Sequence
  synthetic peptide
TKSPFEQHI

SEQ ID NO: 126 - protein, 9 aa, Artificial Sequence
  synthetic peptide
TESPFEQHI

(19) United States
(12) Patent Application Publication
(10) Pub. No.: US 2023/0405101 A1
(43) Pub. Date: Dec. 21, 2023

(54) MOTIF NEOEPITOPES FOR CANCER IMMUNOTHERAPY

(71) Applicant: THE REGENTS OF THE UNIVERSITY OF CALIFORNIA, OAKLAND, CA (US)

(72) Inventors: Edward B. Garon, LOS ANGELES, CA (US); Amy L. Cummings, LOS ANGELES, CA (US); Maura Rossetti, LOS ANGELES, CA (US)

(73) Assignee: THE REGENTS OF THE UNIVERSITY OF CALIFORNIA, OAKLAND, CA (US)

(21) Appl. No.: 18/248,189

(22) PCT Filed: Oct. 6, 2021
(86) PCT No.: PCT/US21/71754

§ 371 Date: Apr. 6, 2023

Related U.S. Application Data
(60) Provisional application No. 63/088,385, filed on Oct. 6, 2020.

(51) Int. Cl. A61K 39/00; A61P 35/00; A61K 39/395

(52) U.S. Cl. CPC A61K 39/0011; A61P 35/00; A61K 39/3955; A61K 2039/5154; A61K 2039/5158; A61K 2039/53

(57) ABSTRACT

Identifying immune checkpoint blockade (ICB) responsive cancer comprises sequencing nucleic acid from a biological sample; and identifying the cancer as ICB responsive when the sequencing detects a nucleic acid encoding a neoepitope that is a nonamer comprising a radical substitution in the second position. This enables the selection of a treatment strategy that improves the efficacy of ICB, based on the patient's profile. The patient can be treated with an agent that enhances responsiveness to ICB, by altering the subject's motif epitope profile or by administering a sensitizing agent. Treating can be with antigen presenting cells (APCs) trained with a neoepitope associated with the subject's cancer. For subjects expressing the HLA supertype B44, the radical substitution consists of a negatively charged amino acid, while for subjects expressing the HLA supertype B27, the radical substitution consists of a positively charged amino acid.

This application claims benefit of U.S. provisional patent application No. 63/088,385, filed Oct. 6, 2020, the entire contents of which are incorporated by reference into this application.

### STATEMENT REGARDING FEDERALLY SPONSORED RESEARCH

This invention was made with government support under Grant Number CA208403, awarded by the National Institutes of Health. The government has certain rights in the invention.

### REFERENCE TO A SEQUENCE LISTING SUBMITTED VIA EFS-WEB

The content of the ASCII text file of the sequence listing named “UCLA281_seq” which is 24 kb in size was created on Oct. 5, 2021, and electronically submitted via EFS-Web herewith the application is incorporated herein by reference in its entirety.

### BACKGROUND

Immune checkpoint blockade (ICB) removes inhibitory signals of T-cell activation, which enables tumor-reactive T cells to overcome regulatory mechanisms and mount an effective antitumor response. These normal regulatory mechanisms maintain immune responses within a physiologic range and protect against autoimmunity. ICB as a therapeutic strategy for treating cancer can induce durable responses across multiple types of cancer. Unfortunately, the responses to these immunotherapies have been limited to a minority of patients and indications.

There remains a need for improved immunotherapy outcomes, particularly in view inconsistent HLA-related ICB outcomes.

### SUMMARY

The methods described herein provide new tools for improved cancer immunotherapy through the identification and use of motif neoepitopes. In one embodiment, described herein is a method of identifying a cancer as responsive to immune checkpoint blockade (ICB). In one embodiment, the method comprises: (a) obtaining a biological sample of the cancer from a subject; (b) sequencing nucleic acid from the biological sample; and (c) identifying the cancer as ICB responsive when the sequencing detects a nucleic acid encoding a neoepitope, wherein the neoepitope is a nonamer comprising a radical substitution in the second position. This identification of cancer that is ICB responsive enables the selection of patients who are good candidates for ICB therapeutic strategies. Moreover, when the cancer is identified as not responsive to ICB, the method enables the development of a treatment strategy that improves the efficacy of ICB, taking into account that individual patient's profile. For example, in some embodiments, the patient can be treated with an agent that induces and/or facilitates responsiveness to ICB, either by altering the subject's motif epitope profile or by administering a sensitizing agent.

Described herein is a method of treating cancer in a subject. In one embodiment, the method comprises administering to the subject a neoepitope associated with the subject's cancer, or a nucleic acid construct encoding the neoepitope. In one embodiment, the method comprises administering to the subject antigen presenting cells (APCs) that have been trained with a neoepitope associated with the subject's cancer. In one embodiment, the method comprises (a) obtaining antigen presenting cells (APCs); (b) pulsing the APCs with a neoepitope associated with the subject's cancer; and (c) administering the pulsed APCs to the subject. The neoepitope is a nonamer comprising a radical substitution in the second position.

In some embodiments, the subject expresses the human leukocyte antigen (HLA) supertype B44 and/or B27. In some embodiments, the subject expresses the HLA supertype B44, and wherein the radical substitution consists of a negatively charged amino acid. In some embodiments, the subject expresses the HLA supertype B27, and the radical substitution consists of a positively charged amino acid. In some embodiments, the negatively charged amino acid is glutamic acid or aspartic acid. In some embodiments, the negatively charged amino acid is glutamic acid. In some embodiments, the positively charged amino acid is histidine, lysine, or arginine.

In some embodiments, the neoepitope associated with the subject's cancer comprises an amino acid sequence encoded by a nucleic acid sequence obtained by sequencing a biological sample obtained from the subject. In some embodiments, the biological sample is a tumor specimen. In some embodiments, the biological sample comprises circulating tumor DNA (ctDNA).

In some embodiments, the cancer is non small cell lung cancer (NSCLC). In some embodiments, the cancer is melanoma. In some embodiments, the cancer is cancer of the head and/or neck. In some embodiments, the APCs are dendritic cells. The APCs can be, for example, matured into dendritic cells. In some embodiments, the APCs are autologous.

In some embodiments, the method further comprises administering to the subject a PD-1 inhibitor. In some embodiments, the methods further comprise administering a second anti-cancer therapy. In certain aspects, the second anti-cancer therapy is a DNA damage checkpoint inhibitor, a chemotherapy, a radiation therapy, a hormonal therapy, a targeted therapy, an immunotherapy or a surgical therapy.

### BRIEF DESCRIPTION OF THE DRAWINGS

FIGS. 1A-1B. Survival based on B44 supertype in UCLA NSCLC cohort. OS—overall survival, PFS—progression-free survival. Survival estimated using the Kaplan-Meier method and compared with a non-parametric log-rank test. Dashed lines represented median. B44 supertype includes all patients with at least one B44 allele. (1A) Patients with at least one B44 allele had a median OS 9.3 months (95% CI 3.9-18.7) vs 18.8 months (95% CI 9.2-38.8), P=0.024. (1B) Patients with at least one B44 allele had a median PFS 2.1 months (95% CI 1.9-4.9) vs 10.2 months (95% CI 5.5-14.5), P=0.040.

FIG. 2. Overall schema. To test the hypothesis that mutational signature dictates radical amino acid substitutions, which in turn influences the likelihood of HLA-B motif neoepitopes (amino acid nonamers inferred from nonsynonymous mutations) and response to immune checkpoint blockade, PBMC were used to identify HLA type 1 alleles, and patients were grouped by HLA-B supertype. PMBC and tumor samples were then used to identify nonsynonymous DNA mutations, amino acid substitutions, and predicted HLA-B neoepitopes. HLA-B neoepitopes were assessed based on motif in silico and in vitro (putative neoantigen refers to peptide form of neoepitopes) and used to compare survival outcomes in varied cohorts. +—present, −—absent, AA—amino acid, HLA—human leukocyte antigen, IC50—half maximal inhibitory concentration, ICB—immune checkpoint blockade, NSCLC—non-small cell lung cancer, P—probability, PBMC—peripheral blood mononuclear cells, TI—transition, TV—transversion. Standard single letter notation used for amino acids. An example of B44 HLA and neoantigen binding is shown (3KPM). Neoantigen EEYLKWATF (SEQ ID NO: 1) is illustrated, and C-terminus motifs are listed (FWYLIMQVA, SEQ ID NO: 2; and FWYLIMQVAHKR, SEQ ID NO: 3).

FIGS. 3A-3C. Proportion of glutamic acid and radical charged substitutions by histology. MEL—melanoma, NSCLC—non-small cell lung cancer. Boxplots summarize patient-level proportions (NSCLC N=38, MEL N=14). Solid lines represent the median, black points appear next to outliers greater than 1.5 times the interquartile range. (3A) On average, 2.8% of substitutions in NSCLC were to glutamic acid vs 5.9% in melanoma, P<0.001. (3B) On average, 74.0% of charged radical substitutions were to positively charged amino acids in NSCLC vs 70.9% in melanoma, P=0.044. (3C) On average, 12.8% of charged radical substitutions were to glutamic acid in NSCLC vs 23.1% in melanoma, P<0.001.

FIGS. 4A-4D. Predicted differences in half-maximal inhibitory concentrations (IC50) of B44 neoepitopes. IC50—half-maximal inhibitory concentration, MT—neoepitope (mutant), WT—wildtype epitope, MT IC50-WT IC50—difference between predicted mutant and wildtype binding, pM—picomolar. Boxplots summarize distribution of points with solid lines representing the median proportion, black points appear next to outliers greater than 1.5 times the interquartile range. Each point represents a neoepitope prediction. B44 motif present refers to neoepitopes featuring a radical substitution to glutamic acid in the anchor position with a known C-terminus (FWYLIMQVA). All predicted neoepitopes had an IC50≤500 nM. (4A) Predicted B44 neoepitope IC50, Wilcoxon test of difference between neoepitopes with and without B44 motif P=0.67. (4B) Predicted B44 neoepitope differences based on percentile rank, Wilcoxon test of difference between neoepitopes with and without B44 P=0.05. (4C) Predicted B44 wildtype epitope IC50, Wilcoxon test of difference between associated neoepitopes with and without B44 motif P<0.001. (4D) Predicted B44 neoepitope IC50 differences between mutant and wildtype peptides (MT IC50—WT IC50) based on motif, Wilcoxon test of difference P<0.001.

FIGS. 5A-5B. Experimental differences in half-maximal inhibitory concentrations (IC50) in HLA-B*18:01 and HLA-B*40:02 predicted neoepitopes based on motif. MTIC50 (nM)—putative neoantigen (mutant) half-maximal inhibitory concentration in nanomolar, MTIC50-WTIC50 (pM)—difference between mutant and wildtype peptide binding in picomolar. Boxplots summarize distribution of points with solid lines representing the median proportion, black points appear next to outliers greater than 1.5 times the interquartile range. Each point represents a peptide. Best reflects mutant peptides with lowest IC50 rank. Motif peptides are defined by a radical charged substitution to glutamic acid in the second position and a known C-terminus (FWYLIMQVA). (5A) B44 competition assay (B*18:01, B*40:02) comparing predicted peptides based on mutant IC50. Wilcoxon test of difference between motif and non-motif peptides P=0.21, best and motif peptides P=0.19. (5B) B44 competition assay (B*18:01, B*40:02) comparing predicted peptides based on difference between mutant and wildtype binding (MT IC50—WT IC50). Wilcoxon test of difference between motif and non-motif peptides and P=0.026, best and motif peptides P=0.016.

FIGS. 6A-6B. Experimental differences in half-maximal inhibitory concentrations (IC50) in HLA-B*40:02 based on charge of artificial and predicted neoepitopes. −/+ relates to strength of amino acid charge based on average side chain pKa in protein conformation in comparison to blood pH: D (3.5±1.2)—negative (−), E (4.2±0.9)—negative (−), H (6.6±1.0)—weakly positive (+), K (10.5±1.1)—positive (++), and R (˜12.5)—strongly positive (+++). MTIC50-WTIC50 (pM)—difference between mutant and wildtype binding in picomolar. Standard single letter amino acid notation used for amino acids. Boxplots summarize distribution of points with solid lines representing the median proportion. Each point represents a peptide. (6A) B44 competition assay comparing predicted and artificial peptides featuring radical substitutions in the anchor position. Wilcoxon test of difference between negatively and positively charged amino acids P=0.031. (6B) B44 competition assay comparing predicted and artificial peptides featuring radical substitutions in non-anchor positions. Wilcoxon test of difference between mutant and wildtype binding not significant between negatively and positively charged amino acids, P=0.90.

FIGS. 7A-7B. Survival based on presence of HLA-B44 and/or B27 specific motif neoepitopes in UCLA B44/B27 NSCLC cohort. Survival estimated using the Kaplan-Meier method and compared between groups with a non-parametric log-rank test. Dashed lines represent medians. (7A) Presence of motif neoepitopes associated with a median OS of 18.7 months (95% CI 6.0—NR) compared to 7.0 months (95% CI 0.7-18.4) when motif neoepitopes were absent, P=0.016. (7B) Presence of motif neoepitopes associated with a median PFS of 12.4 months (95% CI 2.1—NR) compared to 2.8 months (95% CI 1.2-12.4) when motif neoepitopes were absent, P=0.005.

FIGS. 8A-8D. Overall survival and progression-free survival in DF-NSCLC and DF-melanoma cohorts by presence of charged motif neoepitopes. Survival estimated using the Kaplan-Meier method and compared between groups with a non-parametric log-rank test. Dashed lines represent medians. B27/B44 motif neoepitopes required a radical substitution in the anchor position with a known C-terminus. (8A) DF-NSCLC motif neoepitopes associated with median OS that was not reached (95% CI 21.8—NR) vs 13.2 months without (95% CI 8.9—NR, P=0.023). (8B) DF-NSCLC motif neoepitopes associated with median PFS 14.4 months (95% CI 5.6—NR) vs 3.0 months without (95% CI 2.8-5.4, P=0.006). Not all patients had PFS censoring data available in the DF-NSCLC cohort leading to smaller numbers in PFS compared to OS categories. (8C) DF-melanoma motif neoepitopes associated with median OS 34.5 months (95% CI 19.8—NR) vs 13.4 months without (95% CI 8.2-28.0, P=0.027). (8D) DF-melanoma motif neoepitopes associated with median PFS 13.4 months (95% CI 5.4-23.2) vs 3.7 months without (95% CI 2.8-6.0, P=0.010).

FIGS. 9A-9C. Expression of motif and non-motif neoepitopes. (9A) Proportion of neoepitopes that are expressed (FPKM>0). Chi-square tests assessed expressed and non-expressed motif and non-motif neoepitopes. LUAD χ2=8.56 (p=0.003), LUSC χ2=0.24 (p=0.62), SKCM χ2=59.7 (p<0.001). (9B) Degree of motif and non-motif neoepitope expression was assessed by Wilcoxon test using RGL method to account for repeat measures. RNA expression was determined with FPKM values. LUAD W=−1.0 (p=0.32), LUSC W=−0.93 (p=0.35), SKCM W=−9.6 (p<0.001). Boxes represent 25th and 75th percentiles, whiskers demonstrate minimum and maximum, horizontal line demonstrates median. (9C) Fold change in expression of neoepitopes was determined as a comparison to the mean expression of individual genes that produce neoepitopes across the cohort by tumor type. Fold change was determined using normalized HTSeq RNA counts. Expression fold change of motif and non-motif neoepitopes was assessed with Wilcoxon test using RGL method. Genes with zero value in all samples were discarded during normalization. LUAD neoepitopes (motif n=159, non-motif n=2465), LUSC neoepitopes (motif n=170, non-motif n=2062), SKCM neoepitopes (motif n=667, non-motif n=4434). Performed in HLA-B44 patients. LUAD n=239, LUSC n=228, SKCM n=227. LUAD—lung adenocarcinoma, LUSC—lung squamous cell carcinoma, SKCM—cutaneous melanoma, IQR—interquartile range, FPKM—Fragments Per Kilobase Million, RGL—Rosner-Glynn-Lee.

FIGS. 10A-10D. Antigen presentation machinery gene mutations by histology. (10A) Proportion of patients with at least one APM gene mutation by tumor type. Chi-square tests evaluated presence of mutated APM genes with presence of motif neoepitopes, LUAD χ2=2.3 (p=0.13), LUSC χ2=3.89 (p=0.048), SKCM χ2=6.59 (p=0.01). (10B) Mutated APM genes in LUAD. (10C) Mutated APM genes in LUSC. (10D) Mutated APM genes in SKCM. Individual tumors can have more than one mutation in 10B, 10C, and 10D. Performed in HLA-B44 patients. LUAD motif present n=100, motif absent n=139; LUSC motif present n=95, motif absent n=133; SKCM motif present n=156, motif absent n=71. LUAD—lung adenocarcinoma, LUSC—lung squamous cell carcinoma, SKCM—cutaneous melanoma, APM—antigen presentation machinery.

FIGS. 11A-11E. Immune inhibitory cell and cytokine enrichment by tumor type. (11A) Tregs enrichment by tumor type. (11B) M2 macrophage enrichment by tumor type. Cell enrichment score determined by gene set enrichment analysis in xcell software. Evaluation performed with Wilcoxon tests in HLA-B44 patients with and without motif neoepitopes. Horizontal lines represent median, 25th and 75th percentiles. (11C)-(11E): Cytokine expression levels in HLA-B44 patients with and without motif neoepitopes. Comparisons performed by Wilcoxon tests. Median represented by horizontal boxplot line, boxes represent 25th and 75th percentiles, whiskers demonstrate minimum and maximum. RNA read counts presented as normalized HTSeq counts with log 2 transformation. 11C=LUAD, 11D=LUSC, 11E=SKCM. LUAD motif present n=100, motif absent n=139; LUSC motif present n=95, motif absent n=133; SKCM motif present n=156, motif absent n=71. LUAD—lung adenocarcinoma, LUSC—lung squamous cell carcinoma, SKCM—cutaneous melanoma.

FIGS. 12A-12G. Immune checkpoint gene expression. (12A)-(12C): Expression of immune checkpoint genes in HLA-B44 patients with and without motif neoepitopes. Comparisons performed by Wilcoxon tests. 12A=LUAD, 12B=LUSC, 12C=SKCM. (12D)-(12F): Correlation between PD-L1 RNA counts and RPPA by linear regression. Shaded area represents 95% confidence interval. 12D=LUAD, 12E=LUSC, 12F=SKCM. 12G: Difference in expression of PD-L1 by RPPA in patients with and without motif neoepitopes assessed by Wilcoxon tests. Median represented by horizontal boxplot line, boxes represent and 75th percentiles, whiskers demonstrate minimum and maximum. RNA read counts presented as normalized HTSeq counts with log 2 transformation. LUAD motif present n=100, motif absent n=139; LUSC motif present n=95, motif absent n=133; SKCM motif present n=156, motif absent n=71. LUAD—lung adenocarcinoma, LUSC—lung squamous cell carcinoma, SKCM—cutaneous melanoma, RPPA—Reverse Phase Protein Array.

FIGS. 13A-13F. Overall survival and progression free survival based on presence of motif neoepitope and PD-L1 status. Survival was assessed in HLA-B44 patients treated with single-agent anti-PD-1. Survival estimated with Kaplan-Meier method and nonparametric log-rank tests were used for between group comparison. Risk tables show number at risk per time interval. Tick marks represent censoring. Hazard ratios were estimated using proportional hazards. PD-L1 H defined as TPS cutoff of ≥50%. (13A) Motif present, PD-L1 H median OS NR (95% CI 18-NR); Motif present, PD-L1 L median OS 14 months (95% CI 6.0-NR); Motif absent, PD-L1 H median OS 19 months (95% CI 13-NR); Motif absent, PD-L1 L median OS 15 months (95% CI 8-NR). (13B) Motif present, PD-L1 H median PFS 31 months (95% CI 11-NR); Motif present, PD-L1 L median PFS 3.5 months (95% CI 2.1-NR); Motif absent, PD-L1 H median PFS 3.4 months (95% CI 1.7-NR); Motif absent, PD-L1 L median PFS 5.2 months (95% CI 2.8-12). (13C) Motif present, PD-L1 H median OS NR (95% CI 18-NR) vs motif absent and/or PD-L1 L median OS 16 months (95% CI 13-27); Univariable HR p=0.078. (13D) Motif present, PD-L1 H median PFS 31 months (95% CI 11-NR) vs motif absent and/or PD-L1 L median PFS 4.5 months (95% CI 2.8-6.8); Univariable HR 0.27, p=0.009. (13E) Cox proportional hazards model for overall survival comparing motif present, PD-L1 high tumors with motif absent and/or PD-L1 low. (13F) Cox proportional hazards model for progression-free survival comparing motif present, PD-L1 high tumors with motif absent and/or PD-L1 low. OS—overall survival, PFS—progression free survival, H—high, L—low, TPS—tumor proportion score, CI—confidence interval, NR—not reached, HR—hazard ratio.

FIG. 14. Overall schema of hypothesized mechanisms of immune evasion in HLA-B44 tumors harboring motif neoepitopes. DNA damage results in HLA-B44 motif neoepitopes [radical negative AA substitutions in the anchor position (e.g. G>E) with well-defined C-terminus (FWYLIVM; SEQ ID NO: 4)]. While immunoediting (top right) may eliminate tumors harboring B44 motif neoepitopes, potential mechanisms of evading immune destruction of these tumors include: 1) Decreased mRNA expression of genes harboring motif neoepitopes.²) Disruption in antigen presentation via increased mutations involving genes associated with APM.³) Induction of immune checkpoints by tumor cells.⁴) Infiltration of immune inhibitory cells within the TME. HLA-B44—Human leukocyte antigen B44 supertype, Neg—negative, AA—amino acid, APM—antigen presentation machinery, TME—tumor microenvironment, M2-M2 macrophage, Tregs—Regulatory T cell.

FIG. 15. Correlation between frequency of motif neoepitopes and immune inhibitory cell enrichment. Frequency of motif neoepitopes occurring in each patient was compared to enrichment scores for M2 macrophages and Tregs using linear regression and Pearson's coefficient test.

FIG. 16A-16C. Immune cell enrichment by tumor type. Immune cell enrichment in HLA-B44 patients with and without motif neoepitopes. Cell enrichment score determined by gene set enrichment analysis in xcell software. Evaluation performed with Wilcoxon tests. Horizontal lines represent median, 25th and 75th percentiles. (16A)=LUAD, (16B)=LUSC, (16C)=SKCM. LUAD motif present n=100, motif absent n=139; LUSC motif present n=95, motif absent n=133; SKCM motif present n=156, motif absent n=71. LUAD—lung adenocarcinoma, LUSC—lung squamous cell carcinoma, SKCM—cutaneous melanoma.

FIG. 17. Correlation between frequency of motif neoepitopes and immune checkpoint gene expression. Frequency of motif neoepitopes occurring in each patient was compared to immune checkpoint gene expression using linear regression and Pearson's coefficient test.

FIGS. 18A-18D. Overall survival and progression free survival based on presence of motif neoepitope and TMB status. Survival was assessed in HLA-B44 patients treated with single-agent anti-PD-1. Survival estimated with Kaplan-Meier method and nonparametric log-rank tests were used for between group comparison. Risk tables show number at risk per time interval. Tick marks represent censoring. TMB-H defined as ≥10 mutations/megabase (mut/Mb). (18A) Motif Present, TMB High median OS 28.2 months (95% CI 16.2—NR), Motif Present, TMB Low median OS 13.5 months (95% CI 3.9—NR), Motif Absent, TMB High median OS 18.4 months (95% CI 7.0—NR), Motif Absent, TMB Low median OS 16.2 months (95% CI 13.2—NR). (18B) Motif Present, TMB High median PFS 31.3 months (7.3—NR), Motif Present, TMB Low median PFS 3.5 months (95% CI 2.1—NR), Motif Absent, TMB High median PFS 6.3 months (95% CI 2.9—NR), Motif Absent, TMB Low median PFS 4.2 months (95% CI 2.7-7.8). (18C) Motif Present, TMB High median OS 28.2 months (95% CI 16.2—NR), Motif Absent and/or TMB-L median OS 15.9 months (95% CI 9.0-35.1), HR 0.56, p=0.2. (18D) Motif Present, TMB High median PFS 31.3 months (7.3—NR), Motif Absent and/or TMB-L median PFS 4.18 months (95% CI 2.76-6.38), HR 0.21, p=0.002. OS—overall survival, PFS—progression free survival, H—high, L—low, TMB—tumor mutational burden, CI—confidence interval, NR—not reached.

FIG. 19. Levels of immune escape in the setting of motif neoepitopes by tumor type. We observed different types and degrees of immune escape mechanisms among histologies. LUAD displayed depletion of motif neoepitope-producing mutations at the DNA level and greatest degree of immune checkpoint induction. LUSC showed mutations in APM genes, enrichment of immune inhibitory cells in the TME, and induction of immune checkpoints. SKCM demonstrated decreased mRNA expression of genes harboring motif neoepitopes, APM gene mutations and induction of immune checkpoints. *Statistical significance not maintained when accounting for TMB-H. **Statistical significance not maintained when accounting for multiple hypothesis testing. LUAD—lung adenocarcinoma, LUSC—lung squamous cell carcinoma, SKCM—cutaneous melanoma, APM—antigen presentation machinery, TME—tumor microenvironment, M2—M2 macrophage, Treg—T regulatory cell, TMB-H—Tumor mutational burden high.

### DETAILED DESCRIPTION

The invention provides new methods for cancer immunotherapy that provide improved efficacy for patients based on their individual biological makeup. Individualized therapy leads to an enhanced immune response through the use of antigen presenting cells trained to recognize neoepitopes present in the patient's tumor.

### Definitions

All scientific and technical terms used in this application have meanings commonly used in the art unless otherwise specified. As used in this application, the following words or phrases have the meanings specified.

Motif neoepitopes require the presence of a predicted nonamer with a radical substitution in the anchor (2nd) position and C-terminus that matched supertype motif. B27 motif neoepitopes required a radical mutation substituting a positively charged amino acid in the 2nd position of a predicted nonamer; B44 motif neoepitopes required a radical glutamic acid in the 2nd position of a nonamer. An aspartic acid, another negative amino acid, in the second position of a nonamer in place of glutamic acid.

As used herein, a “radical substitution” means a substitution of an amino acid that results in a different charge (positive or negative) relative to the wild type.

As used herein, “antigen-presenting cell” or “APC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells are a preferred type of antigen presenting cell. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of expressing the encoded protein.

The term “primer,” as used herein, means an oligonucleotide designed to flank a region of DNA to be amplified. In a primer pair, one primer is complementary to nucleotides present on the sense strand at one end of a polynucleotide fragment to be amplified and another primer is complementary to nucleotides present on the antisense strand at the other end of the polynucleotide fragment to be amplified. A primer can have at least about 11 nucleotides, and preferably, at least about 16 nucleotides and no more than about 35 nucleotides. Typically, a primer has at least about 80% sequence identity, preferably at least about 90% sequence identity with a target polynucleotide to which the primer hybridizes.

As used herein, the term “probe” refers to an oligonucleotide, naturally or synthetically produced, via recombinant methods or by PCR amplification, that hybridizes to at least part of another oligonucleotide of interest. A probe can be single-stranded or double-stranded.

As used herein, the term “active fragment” refers to a substantial portion of an oligonucleotide that is capable of performing the same function of specifically hybridizing to a target polynucleotide.

As used herein, “hybridizes,” “hybridizing,” and “hybridization” means that the oligonucleotide forms a noncovalent interaction with the target DNA molecule under standard conditions. Standard hybridizing conditions are those conditions that allow an oligonucleotide probe or primer to hybridize to a target DNA molecule. Such conditions are readily determined for an oligonucleotide probe or primer and the target DNA molecule using techniques well known to those skilled in the art. The nucleotide sequence of a target polynucleotide is generally a sequence complementary to the oligonucleotide primer or probe. The hybridizing oligonucleotide may contain nonhybridizing nucleotides that do not interfere with forming the noncovalent interaction. The nonhybridizing nucleotides of an oligonucleotide primer or probe may be located at an end of the hybridizing oligonucleotide or within the hybridizing oligonucleotide. Thus, an oligonucleotide probe or primer does not have to be complementary to all the nucleotides of the target sequence as long as there is hybridization under standard hybridization conditions.

The term “complement” and “complementary” as used herein, refers to the ability of two DNA molecules to base pair with each other, where an adenine on one DNA molecule will base pair to a guanine on a second DNA molecule and a cytosine on one DNA molecule will base pair to a thymine on a second DNA molecule. Two DNA molecules are complementary to each other when a nucleotide sequence in one DNA molecule can base pair with a nucleotide sequence in a second DNA molecule. For instance, the two DNA molecules 5′-ATGC and 5′-GCAT are complementary, and the complement of the DNA molecule 5′-ATGC is 5′-GCAT. The term complement and complementary also encompasses two DNA molecules where one DNA molecule contains at least one nucleotide that will not base pair to at least one nucleotide present on a second DNA molecule. For instance, the third nucleotide of each of the two DNA molecules 5′-ATTGC and 5′-GCTAT will not base pair, but these two DNA molecules are complementary as defined herein. Typically, two DNA molecules are complementary if they hybridize under the standard conditions referred to above. Typically, two DNA molecules are complementary if they have at least about 80% sequence identity, preferably at least about 90% sequence identity.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, P A, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### METHODS OF THE INVENTION

The present disclosure provides methods for improved cancer immunotherapy. In one embodiment, described herein is a method of identifying a cancer as responsive to immune checkpoint blockade (ICB). In one embodiment, the method comprises: (a) obtaining a biological sample of the cancer from a subject; (b) sequencing nucleic acid from the biological sample; and (c) identifying the cancer as ICB responsive when the sequencing detects a nucleic acid encoding a neoepitope, wherein the neoepitope is a nonamer comprising a radical substitution in the second position. This identification of cancer that is ICB responsive enables the selection of patients who are good candidates for ICB therapeutic strategies. Moreover, when the cancer is identified as not responsive to ICB, the method enables the development of a treatment strategy that improves the efficacy of ICB, taking into account that individual patient's profile. For example, in some embodiments, the patient can be treated with an agent that induces and/or facilitates responsiveness to ICB, either by altering the subject's motif epitope profile or by administering a sensitizing agent.

Described herein is a method of treating cancer in a subject. In one embodiment, the method comprises administering to the subject a neoepitope associated with the subject's cancer, either in peptide form, via exposure to APCs presenting such peptides, and/or in the form of a nucleic acid encoding the neoepitope, such as an RNA vaccine. In one embodiment, the method comprises administering to the subject antigen presenting cells (APCs) that have been trained with a neoepitope associated with the subject's cancer. In one embodiment, the method comprises (a) obtaining antigen presenting cells (APCs); (b) pulsing the APCs with a neoepitope associated with the subject's cancer; and (c) administering the pulsed APCs to the subject. Pulsing, or training, APCs can be achieved by exposing the APCs to the neoepitope. The neoepitope comprises a radical substitution in the second position. The neoepitope comprises 8 to 12 amino acids, and is typically an octamer or a nonamer. In some embodiments, the neoepitope is a nonamer.

In some embodiments, the subject expresses the human leukocyte antigen (HLA) supertype B44 and/or B27. In some embodiments, the subject expresses the HLA supertype B44, and wherein the radical substitution consists of a negatively charged amino acid. In some embodiments, the subject expresses the HLA supertype B27, and the radical substitution consists of a positively charged amino acid. In some embodiments, the negatively charged amino acid is glutamic acid or aspartic acid. In some embodiments, the negatively charged amino acid is glutamic acid. In some embodiments, the positively charged amino acid is histidine, lysine, or arginine. In some embodiments, the positively charged amino acid is arginine.

In some embodiments, the neoepitope associated with the subject's cancer comprises an amino acid sequence encoded by a nucleic acid sequence obtained by sequencing a biological sample obtained from the subject. In some embodiments, the biological sample is a tumor specimen. In some embodiments, the biological sample comprises circulating tumor DNA (ctDNA). Thus, one can identify a neoepitope associated with the subject's cancer by isolating cancer-related DNA from a biological sample obtained from the subject, and sequencing the isolated DNA. The biological sample can be blood, serum, plasma, biopsy material, or other specimen that would contain DNA related to the subject's cancer.

In some embodiments, the cancer is non small cell lung cancer (NSCLC). In some embodiments, the cancer is melanoma. In some embodiments, the cancer is cancer of the head and/or neck.

In some embodiments, the APCs are dendritic cells. The APCs can be, for example, matured into dendritic cells. In some embodiments, the APCs are autologous. For example, the APCs can be modified by exposure to the isolated antigen, alone or as part of a mixture, peptide loading, or by genetically modifying the APC to express a polypeptide that includes one or more epitopes.

In some embodiments, the APCs or dendritic cells (DCs) are isolated from peripheral blood of the subject to be treated. The isolated DCs are then incubated with the subject's motif neoepitope(s). In some embodiments, the incubation is performed at 37 degrees C. for 1-8 hours. In some embodiments the incubation is 2-4 hours. In some embodiments, both dendritic cells and T cells are isolated from the subject, for example, from peripheral blood. In some embodiments, the T cells are activated. In some embodiments, the ration of T cells to DCs is 1:3, 1:5, or 1:10. In some embodiments, the cells (DCs and T cells) are co-cultured for 5, 7, 9, or 12 days.

### Compositions

The invention provides compositions that are useful for treating cancer. The compositions can be used to inhibit tumor growth and to kill tumor cells. In one embodiment, the composition is a pharmaceutical composition. The composition can comprise a therapeutically or prophylactically effective amount of a polypeptide, polynucleotide, recombinant virus, APC or immune cell of the invention, including motif neoepitopes as described herein as well as polynucleotides encoding such motif neoepitopes. In some embodiments, the composition comprises an RNA vaccine. An effective amount is an amount sufficient to elicit or augment an immune response, e.g., by activating T cells. In some embodiments, the composition is a vaccine.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The composition of the invention can further comprise one or more adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. Pharmaceutical compositions and vaccines may also contain other compounds, which may be biologically active or inactive. For example, one or more immunogenic portions of other antigens may be present, either incorporated into a fusion polypeptide or as a separate compound, within the composition or vaccine.

A pharmaceutical composition or vaccine may contain DNA encoding one or more of the polypeptides of the invention, such that the polypeptide is generated in situ.

### EXAMPLES

The following examples are presented to illustrate the present invention and to assist one of ordinary skill in making and using the same. The examples are not intended in any way to otherwise limit the scope of the invention.

### Example 1: Mutational Landscape Influences Immunotherapy Outcomes Among Non-Small Cell Lung Cancer Patients with Human Leukocyte Antigen Supertype B44

Human leukocyte antigen (HLA)-B has been recognized as a major determinant of discrepancies in disease outcomes, and recent evidence suggests a role in immune checkpoint blockade (ICB) efficacy. The B44 supertype, which features an electropositive binding pocket that preferentially displays peptides with negatively charged amino acid anchors, associated with improved survival in ICB-treated melanoma. Yet this effect was not seen in ICB-treated non-small cell lung cancer (NSCLC). This Example shows that mutations leading to glutamic acid substitutions occur more often in melanoma than NSCLC based on mutational landscape. The Example also demonstrates stratifying B44 based on the presence of somatic mutations that lead to negatively charged glutamic acid anchors identifies NSCLC patients with ICB benefit similar to that seen in melanoma. These findings can be used to improve assessment of HLA-related outcomes and prediction of ICB benefit in those with B44, representing approximately half of the world's population.

Supplementary Tables and Supplementary Figures referenced in Example 1 can be found at Cummings, A. L., et al. Nat Cancer 1, 1167-1175 (2020). doi.org/10.1038/s43018-020-00140-1.

### INTRODUCTION

HLA class I moieties are found in all nucleated cells and are the scaffolds that present intracellular peptides to CD8+ T-cells.¹ While they have been implicated in immune responses to cancer for decades,^{2,3} there is burgeoning interest in the role of HLA supertypes, which leverage structural features of binding pocket residues to group HLA alleles.⁴⁻⁶ These designations rely in particular on residues that interact with peptide anchors, which generate the majority of HLA-peptide binding energy, intimately relating supertype to antigenic peptide motifs (conserved amino acids in specific positions of presented peptides).⁴⁻⁶

In ICB-treated melanoma, the B44 supertype (B44) associated with greater overall survival, especially in those with increased glycine to glutamic acid (p.G>E) anchor substitutions.^{3,7} (For single letter notation, “g.” refers to genomic information, i.e., DNA bases, while “p.” denotes protein information, i.e., amino acids, represented as wildtype>mutant.) As melanoma and NSCLC have relatively similar prevalence of somatic mutations and response to ICB,^{8,9} it seemed likely that B44 would associate with clinical benefit across histology. Yet in one study of ICB-treated NSCLC featuring targeted sequencing panels, a protective effect was not found.¹⁰ To determine the robustness of this finding, we evaluated the impact of B44 in our NSCLC cohort treated with single-agent pembrolizumab with over five years of follow-up and tissue samples available (N=65). Additionally, we evaluated the mechanistic underpinnings of divergent ICB response in this group, which we validated in publicly available NSCLC and melanoma cohorts.

### Methods

Patients, Response Assessment, and Sample Processing

Of the 67 advanced NSCLC patients treated on clinical trials with single-agent pembrolizumab at the University of California, Los Angeles who consented to tissue-banking, 65 had adequate germline samples and were included in our retrospective survival analysis. Thirty-eight of these patients had matched peripheral blood mononuclear cells (PBMC) and archival tissue with >20% estimated tumor involvement. These PBMC and tissue samples underwent multiplexed paired-end whole-exome sequencing (WES) to a target depth of 100-150× on HiSeq 2000/3000 (IIlumina, San Diego, CA) performed by the UCLA Technology Center for Genomics & Bioinformatics.³¹ Macrodissection was not performed. DNA isolation was performed with DNeasy Blood & Tissue Kit (Qiagen, Germany); exon capture and library preparation used the KAPA HyperPrep Kit and Nimblegen SeqCap EZ Human Exome Library v3.0 (Roche, Switzerland). The code and data that support the findings of this study are or will be made openly available prior to publication.

Comparator and Validation Cohorts

SRP067938 comprised patients from the UCLA melanoma cohort (N=14) and was used for genomic analyses only. The DF-NSCLC and -melanoma cohort (N=52, N=151) included patients from phs000452, phs000980, phs00694, phs001041, phs001565, and SRP067938, excluding five patients included in the UCLA NSCLC cohort.²¹ Limited clinical information was available for all cohorts.^{11,20,21,32,33} Unique patients from TCGA-LUAD (lung adenocarcinoma), LUSC (lung squamous cell carcinoma), and SKCM (melanoma) with clinical information available (N=518, 496, 470) were used for genomic analyses, particularly modeling corrected DNA base proportions as these datasets have been previously well described.^{22,34} Only one TOGA sample was allowed per patient; for patients with more than one sample, the sample including the highest number of VEP annotations that passed all filters was included.

HLA, TMB, and Neoepitope Prediction

For the UCLA NSCLC cohort, HLA type was obtained by aligning germline WES to UCSC hg38 primary assembly reference via Burrows-Wheeler Aligner (BWA) v0.7 filtered by Sequence Alignment/Map tools (SAMtools) v1.7. HLA calling was performed with ATHLATES software.³⁵ Supertype was determined by 2008 criteria and included alleles with an experimentally established motif or B and F pocket exact match with the exception of B*44:05, which does not have a B and F pocket exact match but previously has been included in B44 analyses.^{6,7} The B44 subset was described by Chowell and colleagues and included alleles B*18:01, B*44:02, B*44:03, B*44:05, and B*50:01.⁷ Somatic mutations were identified using Genome Analysis Toolkit (GATK) software v3.8 including MuTect2 requiring 4 calls to confirm a variant.³⁶ Annotations were performed with SnpEff v4.1 and Ensembl Variant Effect Predictor (VEP) v94.^{37,38} Tumor mutation burden (TMB) was calculated by summing the number of protein coding mutations, allowing only one mutation to be counted per reference base position, and dividing this number by 38 to create a mutations/megabase statistic. High TMB was considered the top 40% of observed values per cohort.¹³ Personalized Variant Antigens by Cancer Sequencing (pVAC-Seq) software was used to predict nonamer HLA-B neoepitopes/IC₅₀ from nonsynonymous mutations, reporting only those with a predicted IC₅₀ less than 500 nM.^{39,40} The NetMHC 4.0 algorithm was used for well characterized alleles, NetMHCpan 4.0 for alleles with limited information and otherwise not eligible for NetMHC. HLA and neoepitope prediction for the DF cohorts was performed by Miao and colleagues and is publicly available.²¹ The code used to produce these data is openly available (broadinstitute.org, ensembl.org, pvac-seq.readthedocs.io).

### Definitions: Radical Substitutions, Mutation Types, and Motif Neoepitopes

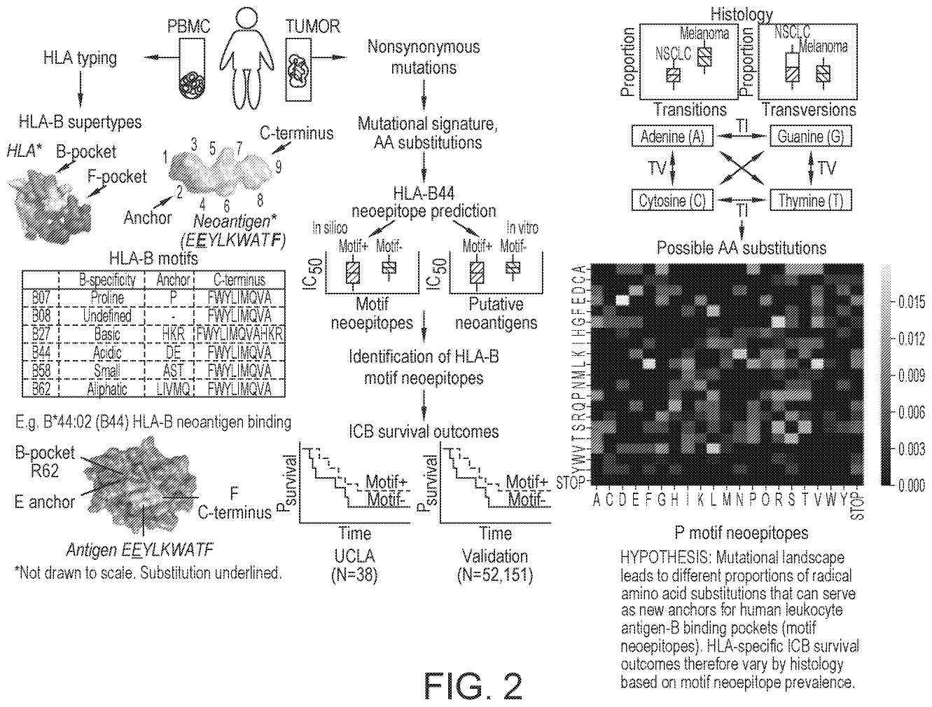

Amino acid charge was based on average side chain pKa in protein conformation.⁴¹ Aspartic acid (D) and glutamic acid (E) were considered negatively charged, histidine (H), lysine (K), and arginine (R) positively charged. Radical substitutions were defined by standard amino acid physiochemical properties including charge, size, hydrophobicity, and polarity;⁴² radical charged substitutions included mutations from oppositely charged or uncharged wildtype amino acid codons to p.D/E (negatively charged) or p.H/K/R (positively charged) codons; stop codons were considered uncharged. Transition mutations were defined as DNA nucleotide mutations between purines (adenine/guanine) or pyrimidines (cytosine/thymine); transversion mutations include all other permutations.^{19,20} Motif neoepitopes were defined by 2008 criteria, requiring the presence of a predicted nonamer with a radical substitution in the anchor (2^{nd}) position and C-terminus that matched supertype motif.^{5,6} B27 motif neoepitopes required a radical mutation substituting a positively charged amino acid in the 2^{nd} position of a predicted nonamer; B44 motif neoepitopes required a radical glutamic acid in the 2^{nd} position of a nonamer (FIG. 2, Table 3). Radical substitutions for B07 anchors, given its specificity for proline, could be any other amino acid; B58 radical substitutions could result from wildtype amino acids other than p.A/S/T; B62 from wildtype amino acids other than p.L/I/V/M/Q. Note survival assessments based on supertype included any patient with the supertype allele, thus patients could be considered in up to two assessments.

Modeling Radical Substitution Proportions Based on Corrected Average DNA Base Proportions

Theoretical amino acid substitutions were based on standard amino acid codons with 192 transitions and 384 transversions possible (including both synonymous and nonsynonymous mutations), 84 of which lead to radical charged substitutions (Supplementary Table 3). Models used to estimate proportions of radical substitutions were created from probability matrices using the amino acids present in the GRCh38 reference updated by corrected average proportions of DNA base mutations specific to each histology based on UCLA NSCLC and melanoma cohort averages. Corrected DNA base proportions were derived from VEP annotation of missense mutations—for every substitution, we checked if the listed DNA base mutation created a theoretically impossible substitution, and if so, updated the mutation call to the complementary DNA base that could cause the substitution (e.g., for p.G>E, if g.C>T was listed as the DNA mutation, it was switched to g.G>A). For amino acid substitutions that could arise from either complementary base pair (e.g., p.S>R can be caused by g.A>C and g.T>G mutations), the original reported DNA mutation was maintained. For visualization purposes, heatmaps depicting amino acid substitution matrices were constructed with Seaborn using Python v3.6 (www.python.org, Beaverton, OR). The code used to produce and compare these models is openly available.

In Vitro Competition Assays

The DUCAF cell line (HLA-B*18:01, B44 supertype) and Sweig007 cell line (HLA-B*40:02, B44 supertype) were cultured in RPMI 1640, supplemented with 2 mM L-Glutamine, 1% penicillin-streptomycin, and 10% Fetal Bovine Serum (FBS). Cultures were maintained between 3×10⁵-2×10⁶ cells/mL. Wildtype and mutant peptides synthesized for HLA-B*18:01 and HLA-B*40:02 competition assays were based on neoepitope prediction per methods above from two patients with B*18:01 (L17, L24) and the only patient with B*40:02 (L47). Neoepitopes were selected based on lowest percentile rank overall, motif, and those featuring radical substitutions with no more than two neoepitopes coming from the same patient in any one category. B*40:02 neoepitopes were used to synthesize wildtype, mutant, and artificial nonamers (Bachem, Torrance, CA; Table 2). Leucine and methionine were selected for uncharged amino acid comparisons given similarity in molecular weight and shape to glutamic acid. All peptides are listed in Table 2. Differences in IC₅₀ were assessed based on a competition-based cellular peptide binding assay.⁴³ For any category assessed with only one representative neoepitope, assays were run twice with both values included in the analysis. Data were acquired on an LSRFortessa Cell Analyzer (BD Biosciences, San Jose CA) and analyses performed with FlowJo v 10.5.0 (Tree Star, Ashland, OR). IC₅₀ were calculated with Prism (GraphPad, San Diego, CA).

Statistical Analysis

Overall survival and progression-free survival were estimated using the Kaplan-Meier method and compared between groups using non-parametric log-rank tests. Hazard ratios (HRs) were estimated using proportional hazards; a hypothesis test based on standard errors compared B44 HRs. Univariable and multivariable analyses were conducted using proportional hazard ratios assessed with chi-square likelihood ratio tests. DNA mutation and amino acid substitution comparisons used standard t-tests for count data and the generalized estimating equations (GEE) method for proportions; Tukey's correction for multiple comparisons was used (SAS v9.4, Cary, NC). Correlations were assessed by Pearson's method. IC₅₀ were compared with Wilcoxon tests. The difference between motif and non-motif neoepitopes gene expression was based on normalized fragments per kilobase million (FPKM) 44 and assessed with a clustered Wilcoxon rank sum test using the Rosner-Glynn-Lee method to account for repeated measures.⁴⁵ All analyses other than GEE were performed with SPSS v24 (Armonk, NY); P-values<0.05 were considered statistically significant and were rounded to the nearest thousandth. Images were created with R v3.6 (www.r-project.org, Vienna, AU).

Results

B44 Associates with Poor Outcomes in NSCLC

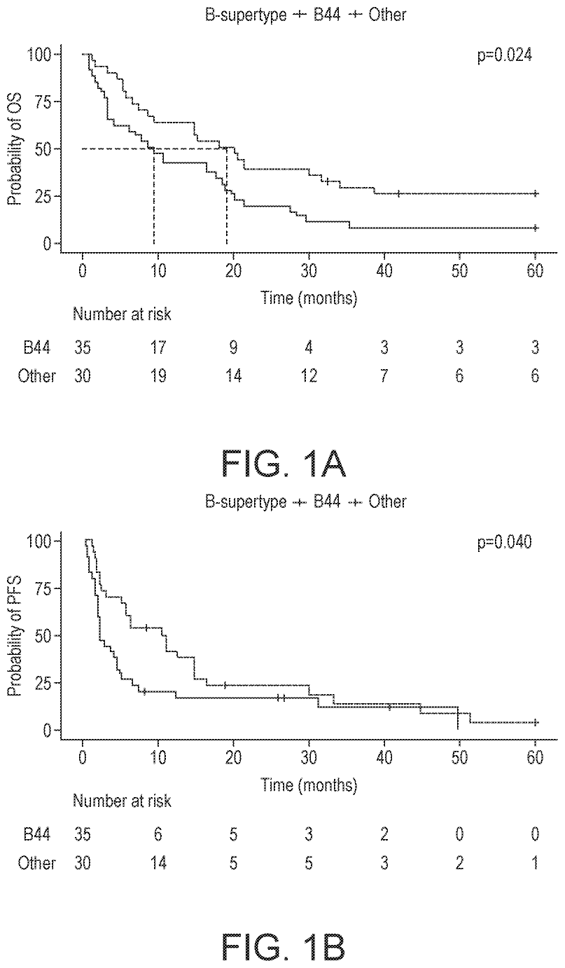

The median age of our UCLA NSCLC cohort was 68 (range 32-91), 63.1% were smokers, and 39% (25/65) were women (Supplementary Table 1). Approximately half (35/65) had at least one B44 allele, with similar prevalence in non-Hispanic white (58.0%, 29/50) and Hispanic white/non-white (40.0%, 6/15) patients. The median overall survival (OS) of B44 patients was 9.3 months (95% CI 3.9-18.7) versus 18.8 months (95% CI 9.2-38.8, P=0.024); median progression-free survival (PFS) was 2.1 months (95% CI 1.9-4.9) versus 10.2 months (95% CI 5.5-14.5, P=0.040) (FIG. 1). The B44 hazard ratio for death (HR) was 2.02 for NSCLC compared to 0.61⁷ for melanoma (P<0.001), suggesting clearly different B44 associations for these cancer types.

Recreating the original B44 subset (HLA-B*18:01, B*44:02, B*44:03, B*44:05, B*50:01) that showed benefit in melanoma patients, 7 it seemed this same group was responsible for B44's risk in NSCLC (Supplementary FIG. 1, Supplementary Table 2). In univariable and multivariable analyses, the B44 subset had HR 2.13-2.88 and associated with worse OS (HR 2.45, P=0.005; univariable P=0.007, multivariable P=0.075) and PFS (HR 4.18, P=0.002; univariable P=0.003, multivariable P=0.096). High programmed death ligand 1 by tumor proportion score (PD-L1 TPS 50%) and adenocarcinoma histology, previously associated with improved ICB survival outcomes,¹¹ were the only protective features (P=0.059 and 0.051 in OS, 0.022 and 0.030 in PFS; see Supplementary Tables 1 and 2 for others¹²⁻¹⁴).

Based on the previously described enrichment of p.G>E anchors in melanoma responders,⁷ we hypothesized that negatively charged glutamic acid substitutions in the anchor position were beneficial for immune presentation due to enhanced binding to B44's positively charged binding pocket.¹⁵⁻¹⁸ We further hypothesized that radical glutamic acid substitutions, or those that substitute glutamic acid for an uncharged or positively charged wildtype amino acid, may be distributed unevenly in melanoma and NSCLC, 8 decreasing the likelihood of substitutions that could act as new B44 anchors in NSCLC tumors. We devised a series of experiments based on whole-exome sequencing (WES) and neoepitope prediction in our NSCLC cohort with adequate tumor tissue available (N=38) to test this hypothesis, which we confirmed in additional cohorts (Methods, FIG. 2).

Radical Glutamic Acid Substitutions Occur More Commonly in Melanoma than NSCLC Based on Mutational Signature

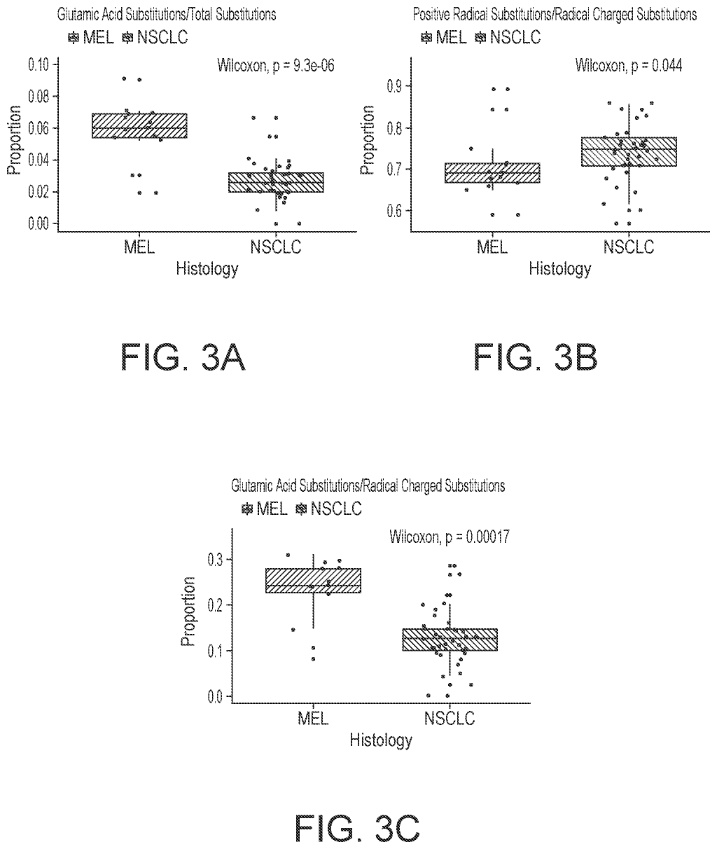

In UCLA cohorts, an average of 2.8% of somatic substitutions were to glutamic acid in NSCLC compared to 5.9% in melanoma (FIG. 3a, P<0.001). We anticipated mutational landscape was responsible for this difference. Melanoma preferentially exhibits transition mutations, particularly cytosine to thymine (g.C>T) mutations due to ultraviolet light dimerization, while NSCLC has relatively more transversion mutations, particularly cytosine to adenine (g.C>A) mutations caused by bulky adducts from smoking.^{19,20} While transition and transversion mutations are equally likely to substitute negatively charged amino acids (4.2% vs 4.2%, P=1.0), transversion mutations are more likely to substitute positively charged amino acids than transition mutations (12.5% vs 6.3%, P=0.021), particularly g.C>A, which decreases radical negative substitutions by 0.2% for every 10% enrichment (Supplementary Table 3).

To model this phenomena, we determined that if all nonsynonymous mutations occurred equally, based on amino acids present in the GRCh38 reference, we would expect 14.0% to result in a radical positive substitution and 5.5% to result in a radical negative substitution (P<0.001), suggesting a natural predisposition for radical positive substitutions. Using corrected average proportions of DNA base mutations for our NSCLC and melanoma cohorts (Methods), we predicted NSCLC would have 12.5% radical positive substitutions and 6.4% radical negative substitutions on average while melanoma would have 11.1% radical positive substitutions and 6.6% radical negative substitutions. Reframing as proportions of radical substitutions, this corresponds to 73.8% of radical charged substitutions being positive in NSCLC versus 71.8% in melanoma. In this model, radical negatively charged amino acid substitutions would be distributed equally between aspartic and glutamic acid with glutamic acid representing 11.0% radical substitutions in NSCLC and 14.1% in melanoma (P<0.001).

Characterizing our NSCLC and melanoma cohort substitutions, 74.0% of radical charged substitutions were positive in NSCLC versus 70.9% in melanoma (FIG. 3b, P=0.044). While 12.8% of radical charged substitutions were to glutamic acid in NSCLC (approximately half of radical negatively charged substitutions), 23.1% were to glutamic acid in melanoma (FIG. 3c, P<0.001). This proportionally correlated with observed g.G>A mutations (38.4% in NSCLC vs 71.3% in melanoma, P<0.001, Table 1) and was supported by p.G>E, which drove glutamic acid enrichment in melanoma (Supplementary FIG. 2). Evaluating DNA mutations leading to radical glutamic acid substitutions, enrichment for radical glutamic acid substitutions in both NSCLC and melanoma was found to correlate with g.G>A mutation proportions (R=0.6, P<0.001) and anti-correlate with g.C>A mutation proportions (R=−0.6, P<0.001) (Supplementary FIG. 3).

TABLE 1
Average proportions of DNA mutations leading to radical substitutions
in UCLA NSCLC and melanoma cohorts by cancer type

| | NSCLC | Melanoma | |
|---|---|---|---|
| | (N = 38) | (N = 14) | Difference |
| g.A > C | 0.020 (0.02) | 0.010 (0.01) | 0.010 |
| g.A > G | 0.086 (0.04) | 0.058 (0.03) | 0.028* |
| g.A > T | 0.007 (0.02) | 0.001 (0.01) | 0.001 |
| g.C > A | 0.207 (0.04) | 0.076 (0.02) | 0.131* |
| g.C > G | 0.085 (0.04) | 0.045 (0.02) | 0.040* |
| g.C > T^{†} | — | — | — |
| g.G > A | 0.384 (0.10) | 0.713 (0.05) | −0.329* |
| g.G > C | 0.053 (0.02) | 0.026 (0.01) | 0.027* |
| g.G > T | 0.037 (0.05) | 0.007 (0.03) | 0.030* |
| g.T > A | 0.048 (0.02) | 0.015 (0.00) | 0.033* |
| g.T > C | 0.028 (0.02) | 0.028 (0.02) | 0.000 |
| g.T > G | 0.037 (0.02) | 0.015 (0.01) | 0.022 |

NSCLC—non-small cell lung cancer.
DNA mutations: g.A > C—adenine to cytosine, g.A > G—adenine to guanine, g.A > T—adenine to thymine, g.C > A—cytosine to adenine, g.C > G—cytosine to guanine, g.C > T—cytosine to guanine, g.G > A—guanine to adenine, g.G > C—guanine to cytosine, g.G > T—guanine to thymine, g.T > A—thymine to adenine, g.T > C—thymine to cytosine, g.T > G—thymine to guanine.
*P < 0.001.
^{†}No radical substitutions are caused by g.C > T (cytosine to guanine) mutations.
Standard deviations are shown in parentheses.

Motif Neoepitopes with Radical Glutamic Acid Substitutions in the Anchor Position Suggest Improved B44 Binding in Silico

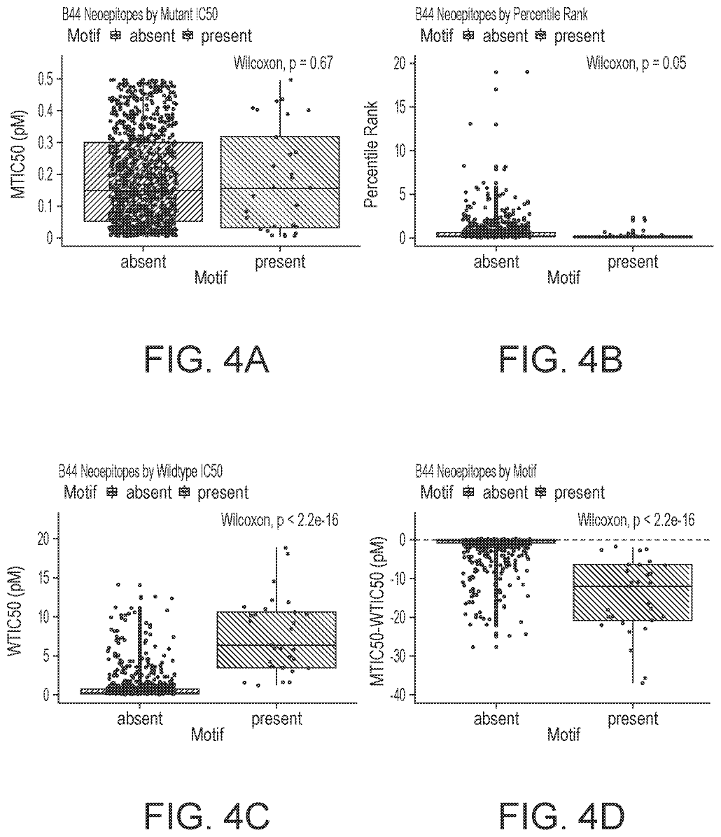

To understand the impact of radical glutamic acid substitutions on B44 binding and motif, we compared all NSCLC B44 neoepitope predictions with half maximal inhibitory concentrations (IC₅₀)≤500 nanomolar (nM). B44 motif neoepitopes were defined based on a radical charged glutamic acid substitution in the anchor position with a known C-terminus (Methods). Among NSCLC B44 cases (N=21), the number of radical glutamic acid substitutions correlated with the number of predicted neoepitopes featuring glutamic acid substitutions (Supplementary FIG. 4, R=0.56, P=0.009), and the number of predicted neoepitopes featuring radical glutamic acid substitutions correlated with the number of B44 motif neoepitopes (Supplementary FIG. 4, R=0.89, P<0.001). A trend towards an association between B44 motif neoepitopes and radical glutamic acid substitutions also was seen (Supplementary FIG. 4, R=0.42, P=0.06). Evaluating B44 motif and non-motif neoepitopes out of all neoepitopes with a predicted IC₅₀≤500 nM, B44 motif neoepitopes did not have significantly different mutant IC₅₀ estimates (FIG. 4a, P=0.67), but trended towards lower percentile rank (FIG. 4b, P=0.05). B44 motif neoepitopes did, however, have wildtype epitopes with significantly higher predicted IC₅₀ (FIG. 4c, P<0.001), suggesting improved mutant binding compared to wildtype (FIG. 4d, P<0.001).

Motif Neoepitopes with Radical Glutamic Acid Substitutions in the Anchor Position Display Enhanced B44 Binding In Vitro

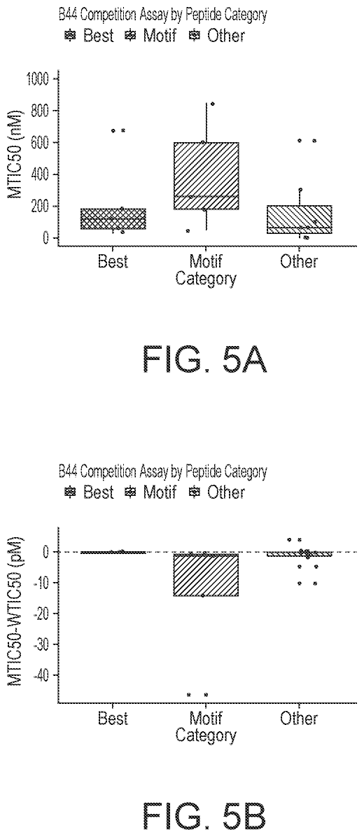

We then assessed mutant and wildtype epitopes in vitro with HLA-B*18:01 and B*40:02 (B44) cell models. Select neoepitopes predicted for NSCLC patients with these alleles were grouped as best (lowest percentile rank), motif, and other (Methods, Table 2). Using competition assays to provide IC₅₀ estimates, there were no significant differences among these groups based on mutant IC₅₀ (FIG. 5a, P=0.21). The difference between mutant and wildtype IC₅₀, however, demonstrated greater comparative binding affinity of B44 motif peptides when compared to all other categories, particularly best predicted peptides (FIG. 5b, P=0.026 and 0.016, respectively), recapitulating the findings of our in silico experiments.

TABLE 2
Experimental peptides

| WT epitope | MT epitope | | | | | | |
|---|---|---|---|---|---|---|---|
| (SEQ ID NOS: | (SEQ ID NOS: | HLA | | MT | MT | WT | |
| 5-24) | 25-58) | Allele | MT | pos | IC₅₀ | IC₅₀ | Category |
| DAQKLLEKM | DEQKLLEKM | B*18:01 | p.A > E | 2 | 0.843 | 2.718 | motif |
| DEELEQMLD | DEELEQMLY | B*18:01 | p.D > Y | 9 | 0.103 | 0.370 | other |
| DQSLIYTLL | DESLIYTLL | B*18:01 | p.Q > E | 2 | 0.259 | 1.071 | motif |
| EELEADTEY | EELEAHTEY | B*18:01 | p.D > H | 6 | 0.062 | 0.206 | best |
| GEVAPSMFL | GEVAPRMFL | B*40:02 | p.S > R | 6 | 0.677 | 0.662 | predicted/best |
| GEVAPSMFL | GEVAPDMFL | B*40:02 | p.S > D | 6 | 0.116 | 0.079 | artificial |
| GEVAPSMFL | GEVAPEMFL | B*40:02 | p.S > E | 6 | 0.142 | 0.079 | artificial |
| HEKVLNEAV | HEKVLNKAV | B*40:02 | p.E > K | 7 | 2.218 | 2.062 | predicted/other |
| HEKVLNEAV | HEKVLNLAV | B*40:02 | p.E > L | 7 | 0.568 | 2.062 | artificial |
| HEKVLNEAV | HEKVLNMAV | B*40:02 | p.E > M | 7 | 0.025 | 0.435 | artificial |
| IAERYGFQY | IEERYGFQY | B*18:01 | p.A > E | 2 | 0.046 | 46.560 | motif |
| KGPSDLLTV | KDPSDLLTV | B*40:02 | p.G > D | 2 | 0.430* | 14.558* | artificial |
| KGPSDLLTV | KEPSDLLTV | B*40:02 | p.G > E | 2 | 0.602* | 14.558* | predicted/motif |
| KGPSDLLTV | KHPSDLLTV | B*40:02 | p.G > H | 2 | 3.580* | 6.821* | artificial |
| KGPSDLLTV | KKPSDLLTV | B*40:02 | p.G > K | 2 | 13.907* | 6.821* | artificial |
| KGPSDLLTV | KRPSDLLTV | B*40:02 | p.G > R | 2 | 26.270* | 6.821* | artificial |
| LALTAPRPY | LELTAPRPY | B*18:01 | p.A > E | 2 | 0.179 | 0.417 | motif |
| LEIDNRLCL | LEIDHRLCL | B*40:02 | p.N > H | 5 | 0.001 | 1.870 | predicted/other |
| MEMFLFFTA | MEDFLFFTA | B*40:02 | p.M > D | 3 | 0.080 | 10.949 | artificial |
| MEMFLFFTA | MEEFLFFTA | B*40:02 | p.M > E | 3 | 0.182 | 10.949 | artificial |
| MEMFLFFTA | MERFLFFTA | B*40:02 | p.M > R | 3 | 0.615 | 10.949 | predicted/other |
| MEPGNNPIF | MESGNNPIF | B*18:01 | p.P > S | 3 | 0.038 | 0.062 | best |
| NGLEIIWAE | NELEIIWAE | B*18:01 | p.G > E | 2 | 0.303 | ^{†} | other |
| QEFWISQAS | QEFWISDAS | B*40:02 | p.Q > D | 7 | 0.006 | 0.002 | artificial |
| QEFWISQAS | QEFWISEAS | B*40:02 | p.Q > E | 7 | 0.009 | 0.002 | artificial |
| QEFWISQAS | QEFWISHAS | B*40:02 | p.Q > H | 7 | 0.003 | 0.002 | predicted/other |
| QENEVLFTM | LENEVLFTM | B*18:01 | p.Q > L | 1 | 0.122 | 0.027 | best |
| RERTMVSTR | RERDMVSTR | B*40:02 | p.T > D | 4 | 14.545 | 22.253 | artificial |
| RERTMVSTR | REREMVSTR | B*40:02 | p.T > E | 4 | 9.448 | 15.344 | artificial |
| RERTMVSTR | RERKMVSTR | B*40:02 | p.T > K | 4 | 10.617 | 15.344 | predicted/other |
| RQFYLWTCL | RHFYLWTCL | B*40:02 | p.Q > H | 2 | 4.760 | 0.909 | other |
| SELLKEFPY | SELLEEFPY | B*18:01 | p.K > E | 5 | 0.186 | 0.111 | best |
| VENSFFLNV | VENSFFLEV | B*40:02 | p.N > E | 8 | 0.649 | 0.798 | other |
| YDGAYAPVL | YEGAYAPVL | B*18:01 | p.D > E | 2 | 0.065 | 0.289 | other |

Standard single letter notation used for amino acids. All predicted neoepitopes had an IC₅₀ ≤ 500 nM.
HLA—human leukocyte antigen, MT—mutant, p.—protein (amino acid), pos—position in peptide (1-9), WT—wildtype. IC₅₀ displayed in picomolars (pM).
*Average of two experimental values included for calculations in which only one neoepitope met criteria for inclusion (see Methods).
^{†} No binding observed, not included in wildtype comparison - also note this peptide does not feature a known C-terminus and does not meet motif criteria.

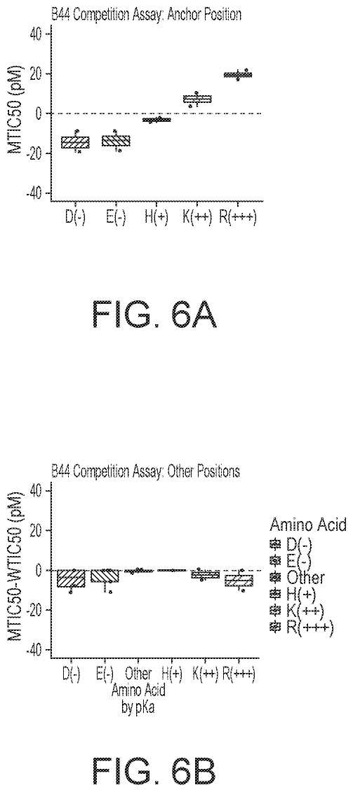

With the B*40:02 cell model, we explored the impact of amino acid charge on IC₅₀ by using all predicted B*40:02 neoepitopes from our cohort that exhibited a radical charged substitution, synthesizing wildtype, mutant, and artificial peptides substituting other charged amino acids in the same position (Table 2). Assessing IC₅₀ differences among oppositely charged artificial and mutant peptides in relation to wildtype peptides, we demonstrated B*40:02 binding affinity was commensurate with an electrostatic gradient: peptides with negatively charged amino acid anchors exhibited significantly improved binding compared to those with positively charged anchors (FIG. 6a, P=0.031). Evaluating radical charged substitutions in positions 1 and 3-9, artificial and mutant B*40:02 peptides did not reveal significant IC₅₀ differences based on charge (FIG. 6b, P=0.90), limiting electrostatic influence on B44 peptide binding to the anchor position.

Motif Neoepitopes Associate with ICB-Survival in Both B44 and B27 Supertypes

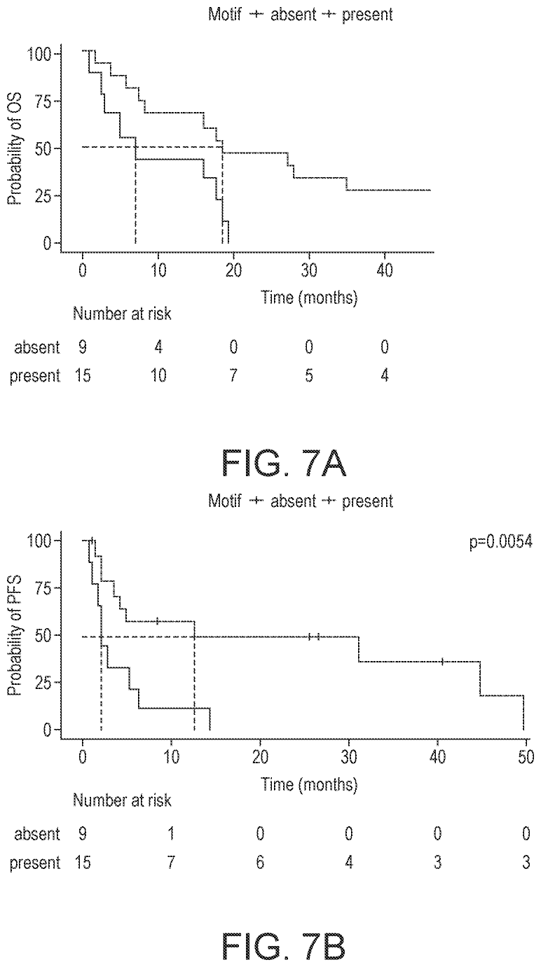

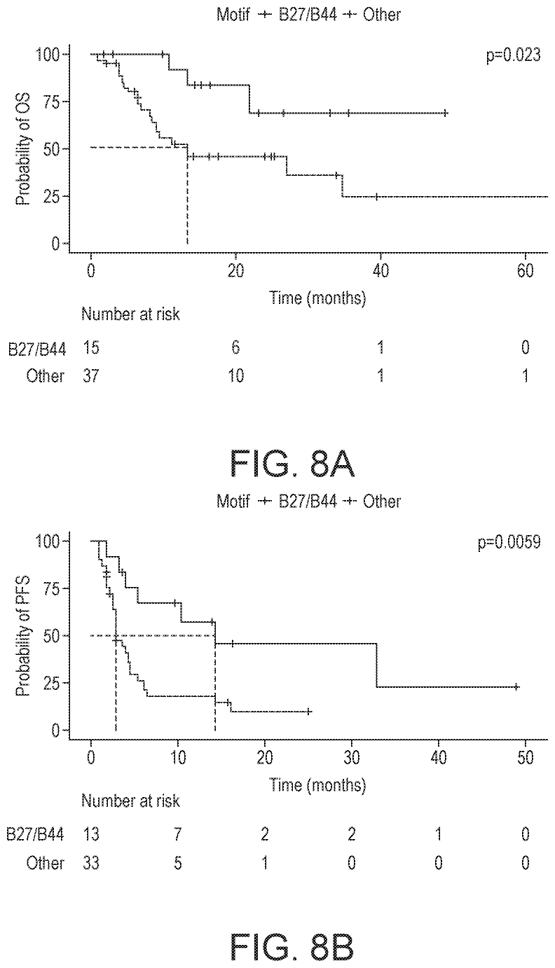

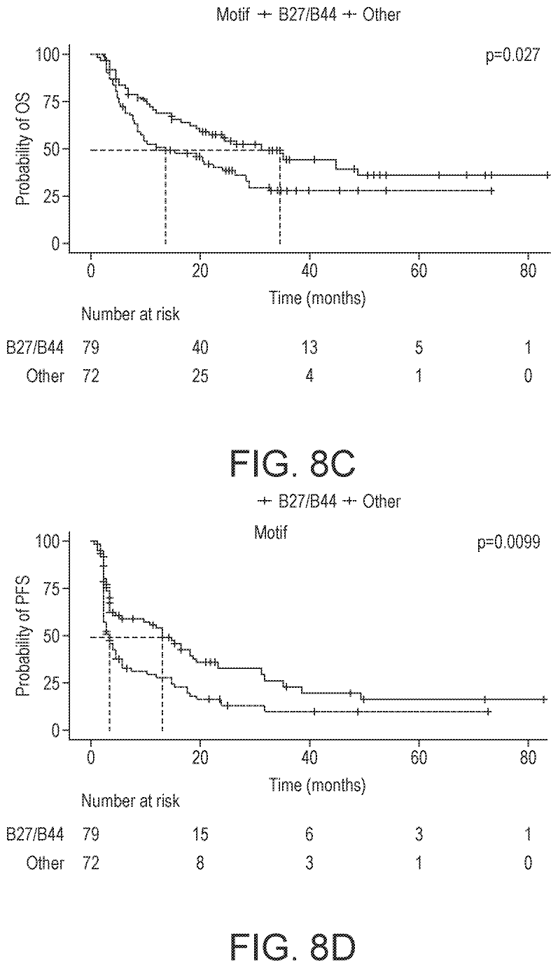

Expanding to our entire cohort, we compared motif and non-motif neoepitopes with radical substitutions as predicted in silico (IC₅₀≤500 nM) for all patients based on supertype (Methods). All HLA-B supertype motif neoepitopes demonstrated enhanced binding affinity in comparison to their wildtype epitope based on predicted IC₅₀ (Supplementary FIG. 5, P<0.001 for all comparisons except B27, P=0.002). Comparing patients with and without at least one predicted motif neoepitope, motif neoepitopes associated with an OS protective effect (HR 0.41, P=0.013) and PFS trend (HR 0.89, P=0.12) (Supplementary FIG. 6). B44 and/or B27 patients represented 53.6% (15/28) of those with motif neoepitopes, and evaluating motif neoepitopes within each of these supertypes suggested the protective effect was driven largely by these groups (Supplementary FIG. 7; B44 OS/PFS P=0.038/0.008, B27 OS/PFS P=0.039/0.062). Markedly, B27 was the only supertype other than B44 that showed a survival benefit (Supplementary FIG. 8) and is the only B-supertype other than B44 that is charged.⁶ Characterizing composite survival outcomes in B44 and/or B27 patients, presence of motif neoepitopes associated with a median OS of 18.7 months (95% CI 6.0—not reached, NR) versus 7.0 months (0.7-18.4, P=0.016) and median PFS of 12.4 months (95% CI 1.2—NR) versus 2.8 months (95% CI 1.2-12.4, P=0.005) (FIG. 7). A list of B44 and B27 motif neoepitopes may be found in Table 3.

TABLE 3
Motif neoepitopes

| | | | | | | | MT epitope | WT epitope | |
|---|---|---|---|---|---|---|---|---|---|
| | | | | | | | (SEQ ID NOS: | (SEQ ID NOS: | WT |
| ID | Chr: position | Gene | Motif | HLA | Sub | P | 59-91) | 92-124) | IC₅₀ |
| L01 | 19: 21294033 | ZNF708 | B44 | B*45:01 | G > E | 2 | CEECGKAFA | CGECGKAFA | 37.6 |
| L02 | 10: 128108409 | MKI67 | B44 | B*44:02 | K > E | 2 | TEWPKRSL | TKQWPKRSL | 18.3 |
| L05 | 17: 18264369 | MIEF2 | B44 | B*40:01 | G > E | 2 | EELAGNLWL | EGLAGNLWL | 24.1 |
| L05 | 15: 24678591 | NPAP1 | B44 | B*40:01 | K > E | 2 | QESDSSFIL | QKSDSSFIL | 5.8 |
| L05 | 2: 162423340 | KCNH7 | B44 | B*44:02 | N > D | 2 | MDMVCMSVF | MNMVCMSVF | 2.9 |
| L08 | 5: 141211193 | PCDHB12 | B44 | B*44:02 | K > E | 2 | NEFKFLKPI | NKFKFLKPI | 18.8 |
| L13 | 11: 105905218 | GRIA4 | B44 | B*44:02 | G > E | 2 | TENVQFDHY | TGNVQFDHY | 22.4 |
| L16* | 2: 129980461 | RAB6C | B44 | B*37:01 | G > E | 2 | EELAGNLWL | RGSDVIITL | 12.4 |
| L16* | 2: 69840988 | GMCL1 | B44 | B*37:01 | K > E | 2 | QESDSSFIL | IKPSRVVAI | 11.7 |
| L17 | 1: 77297600 | AK5 | B44 | B*18:01 | A > E | 2 | TENVQFDHY | IAERYGFQY | 11.4 |
| L17 | 11: 49183163 | FOLH1 | B44 | B*18:01 | A > E | 2 | DEQKLLEKM | DAQKLLEKM | 9.0 |
| L17 | 17: 75831123 | UNC13D | B44 | B*18:01 | K > E | 2 | IEPSRVVAI | PKALHTATF | 20.5 |
| L17 | 20: 9516068 | LAMP5 | B44 | B*18:01 | Q > E | 2 | EEFPYGIEA | SQSELQVFW | 11.6 |
| L17 | 5: 96783147 | ERAP1 | B44 | B*18:01 | Q > E | 2 | EELMELLAA | SQLLLLACV | 9.5 |
| L17 | 5: 141414675 | PCDHGA10 | B44 | B*18:01 | Q > E | 2 | IEERYGFQY | IQGVPLSSY | 7.0 |
| L17 | 5: 181005746 | BTNL3 | B44 | B*18:01 | Q > E | 2 | DESLIYTLL | DQSLIYTLL | 2.1 |
| L21 | 11: 122850063 | BSX | B27 | B*38:01 | L > H | 2 | QHSGLEKRF | QLSGLEKRF | 35.1 |
| L22 | 1: 209801358 | IRF6 | B27 | B*39:01 | D > H | 2 | VHSGLYPGL | VDSGLYPGL | 20.8 |
| L22 | 2: 54844094 | EML6 | B44 | B*44:02 | G > E | 2 | AETQDSEIF | AGTQDSEIF | 6.6 |
| L24 | 1: 74641334 | ERICH3 | B44 | B*18:01 | A > E | 2 | LELTAPRPY | LALTAPRPY | 20.5 |
| L24 | 17: 75831123 | UNC13D | B44 | B*18:01 | K > E | 2 | PEALHTATF | PKALHTATF | 7.0 |
| L24 | 5: 141414675 | PCDHGA10 | B44 | B*18:01 | Q > E | 2 | IEGVPLSSY | IQGVPLSSY | 21.8 |
| L33 | X: 3343810 | MXRA5 | B44 | B*40:01 | V > E | 2 | WEALSVVLI | WGALSVVLI | 2.9 |
| L33 | 2: 68513170 | APLF | B44 | B*40:01 | G > E | 2 | SELEGSTEI | SQLEGSTEI | 8.3 |
| L33 | 19: 51746899 | FPR1 | B44 | B*40:01 | G > E | 2 | LEFAVTFVL | LVFAVTFVL | 29.1 |
| L35 | X: 110173392 | TMEM164 | B44 | B*40:01 | G > E | 2 | AEPLCKYLL | AGPLCKYLL | 23.6 |
| L35 | X: 110173392 | TMEM164 | B44 | B*44:02 | G > E | 2 | AEPLCKYLL | AGPLCKYLL | 20.3 |
| L39 | 16: 30967034 | SETD1A | B44 | B*44:02 | G > E | 2 | REALRLPSF | RGALRLPSF | 8.5 |
| L47 | 8: 69621089 | SULF1 | B44 | B*40:02 | G > E | 2 | KEPSDLLTV | KGPSDLLTV | 16.8 |
| L53 | 11: 55994561 | OR5F1 | B27 | B*27:05 | E > K | 2 | LKLQIILFL | LELQIILFL | 9.8 |
| L53 | X: 19355426 | PDHA1 | B27 | B*27:05 | G > R | 2 | YRMGTSVER | YGMGTSVER | 15.8 |
| L53 | 9: 110375436 | SVEP1 | B27 | B*27:05 | G > R | 2 | NRGRCVAPY | NGGRCVAPY | 17.4 |
| L53 | 15: 42178275 | VPS39 | B44 | B*40:01 | A > E | 2 | METQIQQLL | MATQIQQLL | 16.8 |
| L53 | 3: 172334967 | FNDC3B | B44 | B*40:01 | K > E | 2 | GESPCPSEVL | GKSCPSEVL | 3.0 |

Chr—chromosome, HLA—human leukocyte antigen, MT—mutant, P—peptide position, Sub—amino acid substitution, WTIC₅₀—wildtype IC₅₀ (in mM). Standard single letter notation used for amino acids.
*All substitutions are p.—protein (amino acid). All predicted neoepitopes had an IC₅₀ ≤ 500 nM.

B44 and B27 Motif Neoepitopes Associate with Improved ICB-Survival in NSCLC and Melanoma Validation Cohorts, but are Less Common in NSCLC

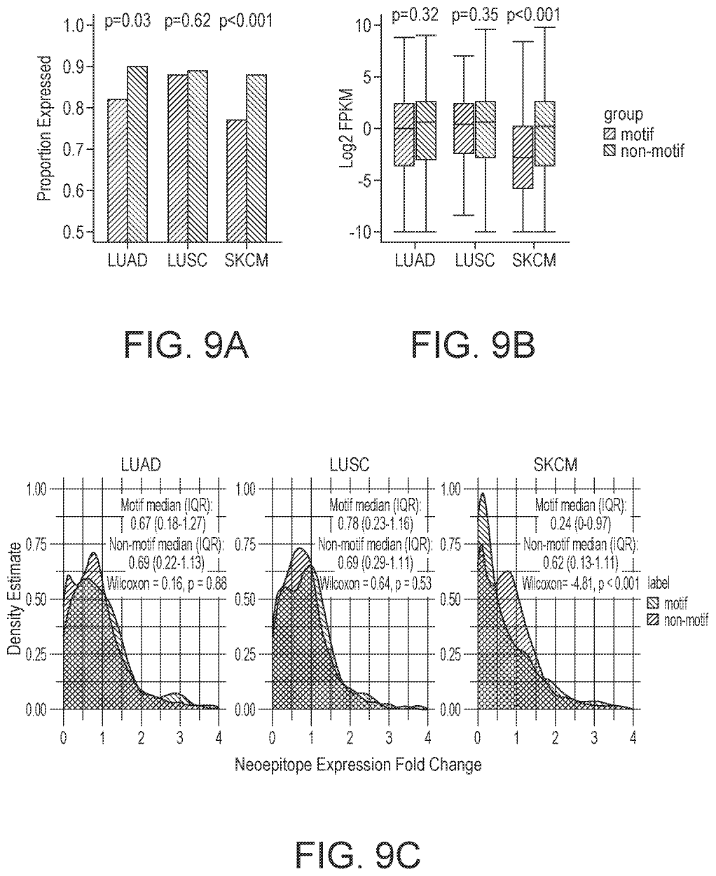

To determine the robustness of the association between motif neoepitopes in charged HLA supertypes with ICB survival outcomes, we validated our findings using publicly available data generated at Dana Farber (DF-NSCLC, DF-melanoma).²¹ Motif neoepitopes were present in 47.1% (16/34) DF-NSCLC B44 and/or B27 patients and 87.9% (80/91) DF-melanoma B44 and/or B27 patients. The presence of B44 and B27 motif neoepitopes was protective in all cohorts (DF-NSCLC OS/PFS P=0.023/0.006; DF-melanoma OS/PFS P=0.027/0.001) (FIG. 8). Further defining these curves, more granular categorization of patients as heterozygous for B44 and B27, B44 (without B27), B27 (without B44), and others suggested that B44 and/or B27 patients without motif neoepitopes had the worst overall survival, followed by patients with uncharged HLA-B, with the best survival outcomes in B44 and/or B27 patients with motif neoepitopes (Supplementary FIG. 9). While we are lacking RNA expression data from these cohorts, based on analysis of The Cancer Genome Atlas (TCGA) melanoma (SKCM) and non-small cell lung cancer adenocarcinoma (LUAD) and squamous cell carcinoma (LUSC) datasets, approximately 85% of patients who exhibited motif neoepitopes at the DNA level also had RNA expression of one or more neoepitopes (Supplementary Table 6).²² However, the degree of gene expression of motif neoepitopes was less than non-motif neoepitopes in all cohorts, suggesting an incentive to the cancer cell to limit motif neoepitope expression. This correlation was particularly strong in the SKCM dataset, the only of the three data sets to include metastatic patients.²² LUAD and LUSC showed subtle differences, but were more similar to each other than SKCM. A summary of TOGA and cohort features may be found in Supplementary Table 7.

### DISCUSSION

In this Example, we identify and resolve the discrepancy in ICB survival based on HLA B44 in NSCLC and melanoma by considering the presence of favorable mutations that can be targeted by the adaptive immune system. Peptides with radical glutamic acid substitutions in the anchor position have enhanced B44 binding, and we find that B44 patients with mutations leading to these substitutions have improved ICB response. Therefore, mutational landscapes that enrich for these mutations, such as those seen in melanoma, are beneficial for B44 patients, while mutational landscapes that are less favorable may lack or show an opposite association, as seen in NSCLC. Our work draws heavily on Chowell and colleagues' contributions to the field, and we believe this investigation supports their prior findings in B44⁷ as well as those in NSCLC.¹⁰ Yet, our analyses offer an opportunity to move beyond histologic associations towards mechanism-informed prognostic markers for patient stratification: while B44, on average, is not predictive of ICB response in NSCLC, it may be for NSCLC patients that exhibit favorable B44 neoepitopes with the converse true for B44 melanoma patients without favorable B44 neoepitopes.

While the relationship of mutational signature to amino acid substitutions has not classically been included in genomic analyses, 23 our data suggest this may be important. Radical amino acid substitutions have significant functional consequence,^{17,24,25} and a number of known neoantigens feature radical substitutions that function as new HLA peptide anchors, including the B44-restricted FAM3C neoantigen (TKSPFEQHI>TESPFEQHI; SEQ ID NOs: 125, 126, respectively) in melanoma.^{3,7,26,27} As is the case with most exploratory analyses, however, our work is limited by its retrospective nature and the availability and quality of patient data. Not all of our patients had tumor specimens that could provide adequate sequencing, and we were more likely to have tissue for patients who had better clinical outcomes, leading to a significant enrichment for durable responders in our WES cohort. Our Kaplan-Meier survival estimations were thus sensitive to the reclassification of individual patients, especially given the long length of follow-up. While we were able to use publicly available cohorts to supplement our findings, especially in melanoma, there is less publicly available long-term survival information available for NSCLC patients treated with single-agent immunotherapy, and certainly even less for other cancer types, which were excluded from this analysis.

Nevertheless, despite different lengths of follow-up and limited patient numbers, we were able to suggest the presence of neoepitopes suggestive of immune intolerance^{28,29} associates with improved immunotherapy outcomes,^{27,30} explaining formerly inconsistent HLA-related ICB outcomes by histology. The focus on B44, in this regard, is not arbitrary. Approximately half of patients have B44, enabling statistically significant conclusions in modestly sized cohorts. Other HLA-B supertypes, such as B27, had significant survival benefits based on the presence of motif neoepitopes, but could not be further defined due to smaller numbers. It is unclear why B27 motif neoepitopes were not more common in NSCLC, especially given the enrichment of positive radical substitutions. NSCLC-specific purifying selection against these mutations is an exciting prospect, but additional evaluation is required.

### CONCLUSION

HLA-based survival associations, specifically those related to ICB outcomes, should be considered in context of histology-specific mutational signature. The identification of motif neoepitopes in patients with B44 associates with ICB survival, and additional validation in other supertypes is desirable.

### REFERENCES

1 Dausset, J. The major histocompatibility complex in man. Science 213, 1469-1474, doi:10.1126/science.6792704 (1981).
2 Algarra, I., Collado, A. & Garrido, F. Altered MHC class I antigens in tumors. Int J Clin Lab Res 27, 95-102 (1997).
3 Chen, D. S. & Mellman, I. Elements of cancer immunity and the cancer-immune set point. Nature 541, 321-330, doi:10.1038/nature21349 (2017).
4 Sidney, J. et al. Several HLA alleles share overlapping peptide specificities. J Immunol 154, 247-259 (1995).
5 Sette, A. & Sidney, J. Nine major HLA class I supertypes account for the vast preponderance of HLA-A and -B polymorphism. Immunogenetics 50, 201-212 (1999).
6 Sidney, J., Peters, B., Frahm, N., Brander, C. & Sette, A. HLA class I supertypes: a revised and updated classification. BMC Immunol 9, 1, doi:10.1186/1471-2172-9-1 (2008).
7 Chowell, D. et al. Patient HLA class I genotype influences cancer response to checkpoint blockade immunotherapy. Science, doi:10.1126/science.aao4572 (2017).
8 Alexandrov, L. B. et al. Signatures of mutational processes in human cancer. Nature 500, 415-421, doi:10.1038/nature12477 (2013).
9 Brahmer, J. R. et al. Safety and activity of anti-PD-L1 antibody in patients with advanced cancer. N Engl J Med 366, 2455-2465, doi:10.1056/NEJMoa1200694 (2012).
10 Negrao, M. V. et al. Journal of thoracic oncology: official publication of the International Association for the Study of Lung Cancer, doi:10.1016/j.jtho.2019.02.008 (2019).
11 Garon, E. B. et al. Pembrolizumab for the treatment of non-small-cell lung cancer. N Engl J Med 372, 2018-2028, doi:10.1056/NEJMoa1501824 (2015).
12 Mok, T. S. et al. Gefitinib or carboplatin-paclitaxel in pulmonary adenocarcinoma. N Engl J Med 361, 947-957, doi:10.1056/NEJMoa0810699 (2009).
13 Hellmann, M. D. et al. Nivolumab plus Ipilimumab in Lung Cancer with a High Tumor Mutational Burden. N Engl J Med 378, 2093-2104, doi:10.1056/NEJMoa1801946 (2018).
14 Chowell, D. et al. Evolutionary divergence of HLA class I genotype impacts efficacy of cancer immunotherapy. Nat Med 25, 1715-1720, doi:10.1038/s41591-019-0639-4 (2019).
15 DiBrino, M. et al. Identification of the peptide binding motif for HLA-B44, one of the most common HLA-B alleles in the Caucasian population. Biochemistry 34, 10130-10138, doi:10.1021/bi00032a005 (1995).
16 Rammensee, H. G., Friede, T. & Stevanoviic, S. MHC ligands and peptide motifs: first listing. Immunogenetics 41, 178-228 (1995).
17 Sinha, N. & Smith-Gill, S. J. Electrostatics in protein binding and function. Curr Protein Pept Sci 3, 601-614 (2002).
18 Macdonald, W. A. et al. A naturally selected dimorphism within the HLA-B44 supertype alters class I structure, peptide repertoire, and T cell recognition. J Exp Med 198, 679-691, doi:10.1084/jem.20030066 (2003).
19 Hodis, E. et al. A landscape of driver mutations in melanoma. Cell 150, 251-263, doi:10.1016/j.cell.2012.06.024 (2012).
20 Rizvi, N. A. et al. Cancer immunology. Mutational landscape determines sensitivity to PD-1 blockade in non-small cell lung cancer. Science 348, 124-128, doi:10.1126/science.aaa1348 (2015).
21 Miao, D. et al. Genomic correlates of response to immune checkpoint blockade in microsatellite-stable solid tumors. Nat Genet 50, 1271-1281, doi:10.1038/541588-018-0200-2 (2018).
22 Cancer Genome Atlas Research, N. et al. The Cancer Genome Atlas Pan-Cancer analysis project. Nat Genet 45, 1113-1120, doi:10.1038/ng.2764 (2013).
23 Vitkup, D., Sander, C. & Church, G. M. The amino-acid mutational spectrum of human genetic disease. Genome Biol 4, R72, doi:10.1186/gb-2003-4-11-r72 (2003).
24 Zuckerkandl, E. & Pauling, L. Molecules as documents of evolutionary history. J Theor Biol 8, 357-366, doi:10.1016/0022-5193(65)90083-4 (1965).
25 Zhang, J. Rates of conservative and radical nonsynonymous nucleotide substitutions in mammalian nuclear genes. J Mol Evol 50, 56-68, doi:10.1007/s002399910007 (2000).
26 Hodi, F. S. et al. Improved survival with ipilimumab in patients with metastatic melanoma. N Engl J Med 363, 711-723, doi:10.1056/NEJMoa1003466 (2010).
27 McGranahan, N. et al. Clonal neoantigens elicit T cell immunoreactivity and sensitivity to immune checkpoint blockade. Science 351, 1463-1469, doi:10.1126/science.aaf1490 (2016).
28 Duan, F. et al. Genomic and bioinformatic profiling of mutational neoepitopes reveals new rules to predict anticancer immunogenicity. J Exp Med 211, 2231-2248, doi:10.1084/jem.20141308 (2014).
29 Teku, G. N. & Vihinen, M. Pan-cancer analysis of neoepitopes. Sci Rep 8, 12735, doi:10.1038/541598-018-30724-y (2018).
30 Snyder, A. et al. Genetic basis for clinical response to CTLA-4 blockade in melanoma. N Engl J Med 371, 2189-2199, doi:10.1056/NEJMoa1406498 (2014).
31 Zaretsky, J. M. et al. Mutations Associated with Acquired Resistance to PD-1 Blockade in Melanoma. N Engl J Med 375, 819-829, doi:10.1056/NEJMoa1604958 (2016).
32 Herbst, R. S. et al. Pembrolizumab versus docetaxel for previously treated, PD-L1-positive, advanced non-small-cell lung cancer (KEYNOTE-010): a randomised controlled trial. Lancet 387, 1540-1550, doi:10.1016/S0140-6736(15)01281-7 (2016).
33 Ribas, A. et al. Association of Pembrolizumab With Tumor Response and Survival Among Patients With Advanced Melanoma. Jama 315, 1600-1609, doi:10.1001/jama.2016.4059 (2016).
34 Kandoth, C. et al. Mutational landscape and significance across 12 major cancer types. Nature 502, 333-339, doi:10.1038/nature12634 (2013).
35 Liu, C. et al. ATHLATES: accurate typing of human leukocyte antigen through exome sequencing. Nucleic Acids Res 41, e142, doi:10.1093/nar/gkt481 (2013).
36 DePristo, M. A. et al. A framework for variation discovery and genotyping using next-generation DNA sequencing data. Nat Genet 43, 491-498, doi:10.1038/ng.806 (2011).
37 Cingolani, P. et al. A program for annotating and predicting the effects of single nucleotide polymorphisms, SnpEff: SNPs in the genome of Drosophila melanogaster strain w1118; iso-2; iso-3. Fly (Austin) 6, 80-92, doi:10.4161/fly.19695 (2012).
38 Zerbino, D. R. et al. Ensembl 2018. Nucleic Acids Res 46, D754-D761, doi:10.1093/nar/gkx1098 (2018).
39 Hundal, J. et al. pVAC-Seq: A genome-guided in silico approach to identifying tumor neoantigens. Genome Med 8, 11, doi:10.1186/s13073-016-0264-5 (2016).
40 Vita, R. et al. The Immune Epitope Database (IEDB): 2018 update. Nucleic Acids Res, doi:10.1093/nar/gkyl006 (2018).
41 Grimsley, G. R., Scholtz, J. M. & Pace, C. N. A summary of the measured pK values of the ionizable groups in folded proteins. Protein Sci 18, 247-251, doi:10.1002/pro.19 (2009).
42 Jencks W P, R. J. Handbook of Biochemistry and Molecular Biology. 4th edn, 595-635 (CRC Press, 2010).
43 Kessler, J. H. et al. Competition-based cellular peptide binding assay for HLA class I. Curr Protoc Immunol Chapter 18, Unit 18 12, doi:10.1002/0471142735.im1812s61 (2004).
44 Mortazavi, A., Williams, B. A., McCue, K., Schaeffer, L. & Wold, B. Mapping and quantifying mammalian transcriptomes by RNA-Seq. Nat Methods 5, 621-628, doi:10.1038/nmeth.1226 (2008).
45 Rosner, B., Glynn, R. J. & Lee, M. L. Extension of the rank sum test for clustered data: two-group comparisons with group membership defined at the subunit level. Biometrics 62, 1251-1259, doi:10.1111/j.1541-0420.2006.00582.x (2006).

### Example 2: Immune Editing and Escape in Non-Small Cell Lung Cancer and Melanoma Tumors with Human Leukocyte Antigen Supertype B44 Motif Neoepitopes

This Example provides evidence for immunoediting and escape in the context of human leukocyte antigen-B44 (HLA-B44) motif-neoepitopes and evaluates response to immune checkpoint blockade (ICB) in those with both motif-neoepitopes and PD-L1 expression. Lung adenocarcinoma (LUAD), lung squamous cell carcinoma (LUSC), and cutaneous melanoma (SKCM) multi-omics from The Cancer Genome Atlas were evaluated. B44 motif-neoepitopes were defined as nonamers with radical negatively charged substitutions in the anchor position. We evaluated neoepitope mRNA expression, antigen presentation machinery (APM) mutations, immune inhibitory cell enrichment, and immune checkpoint gene expression in the presence of motif-neoepitopes. Survival was assessed based on motif-neoepitopes and PD-L1 expression in non-small cell lung cancer (NSCLC) patients on anti-PD-1 treatment.

Presence of B44 in LUAD inversely associated with proportion of negatively-charged substitutions (OR=0.24, p=0.04), suggesting immunoediting. Motif-neoepitope mRNA levels were less than non-motif neoepitopes (p<0.001) in SKCM, putatively representing immune escape. Tumors with motif-neoepitopes had more APM mutations in LUSC (p=0.04) and SKCM (p=0.01), enrichment of M2 macrophages (p=0.01) and regulatory T-cells (p=0.04) in LUSC, and increased expression of immune checkpoints in LUAD (PD-L1, CTLA-4, LAG-3, TIGIT), LUSC (LAG-3, TIM-3), and SKCM (PD-L1, LAG-3) than those without motif-neoepitopes, suggesting immune escape. NSCLC patients with motif-neoepitopes and PD-L1 expression showed improved progression-free survival (HR=0.25, p=0.028), implying these tumors are reliant on PD-L1 induction for evasion.

Tumors with B44 motif-neoepitopes showed evidence for immunoediting and escape, albeit with varying degrees and mechanisms. NSCLC patients with both motif-neoepitopes and PD-L1 expression disproportionately comprise long-term survivors on anti-PD-1 therapy.

### INTRODUCTION

Biomarkers of efficacy for immune checkpoint blockade (ICB) are an area of substantial investigation. The first biomarker to receive approval by the Food and Drug Administration for ICB was immunohistochemical (IHC) assessment of PD-L1.(1) Although predictive of long-term survival,(2) the majority of patients with expression of PD-L1 do not respond to treatment, and conversely, some patients without PD-L1 expression have shown tumor regression with ICB.(3) Tissue-agnostic microsatellite instability (MSI) status has received approval,(4) however, MSI is exceedingly rare in the majority of tumor types.(5) Recently, tumor mutational burden (TMB) was granted accelerated approval given improvement in response rates in TMB-high tumors.(6) While high TMB serves as a surrogate for neoantigen load, TMB alone cannot identify if clinically important neoantigens are present.

Human leukocyte antigen (HLA) binding of neoantigens, however, may provide more clinically relevant insight as a biomarker. HLA class I molecules present processed intracellular peptide fragments to cytotoxic T-cells,(7) alerting the adaptive immune system to the presence of intracellular pathogens and tumor cells.(8) HLA class I alleles are clustered into supertypes based on structural features of B and F binding pockets which determine peptide antigenic specificity, termed the peptide binding motif.(9) The HLA-B44 supertype (B44) has an electropositive binding pocket that favorably binds and displays negatively charged anchor residues.(10) HLA-B has implications in pathophysiology and outcomes of various inflammatory and infectious diseases,(11) and emerging evidence suggests a putative role in predicting efficacy of ICB.

In melanoma patients treated with ICB, B44 was associated with improved overall survival (OS), largely driven by neoepitopes featuring glycine to glutamic acid (G>E) substitutions in the anchor position.(12) However, a protective effect was not seen with B44 in ICB-treated non-small cell lung cancer (NSCLC).(13) Our group demonstrated that B44 patients with motif-matched neoepitopes exhibited improved OS in ICB-treated NSCLC, and discrepancies in mutational landscapes that enrich for glutamic acid substitutions in melanoma explained disparate outcomes between tumor types.(14) Since B44 motif-matched neoepitopes have been shown to bind particularly well to HLA-B44 and associate with improved clinical outcomes, these neoepitopes provide a unique opportunity to study tumors with well-defined mutations that are more likely to be immunogenic.

It is plausible that cancers with favorable neoepitopes to the host's HLA type are less likely to develop based on immune surveillance. Presumably, during tumorigenesis, clones with strongly immunogenic neoantigens are selected against through T-cell mediated immunoediting, resulting in clinically relevant tumors primarily composed of poorly immunogenic cancer clones.(15) Immunoediting was first demonstrated in mouse models in which tumors from immunosuppressed mice regressed after transplantation into immunocompetent subjects, and outgrowth of tumor cells that occurred in immunocompetent mice was attributed to lack or loss of strong neoantigens.(16-18) Early evidence for immunoediting in humans was seen in immunosuppressed solid organ transplant patients who demonstrated higher incidence of malignancies without known viral etiologies, presumably due to lack of immune-mediated selective pressure.(19) Emerging evidence suggests that immunoediting plays a role in shaping the neoantigen landscape of human tumors,(20-22) with a pan-cancer study showing that neoantigen load was depleted relative to expected numbers in samples from The Cancer Genome Atlas (TCGA).(23)

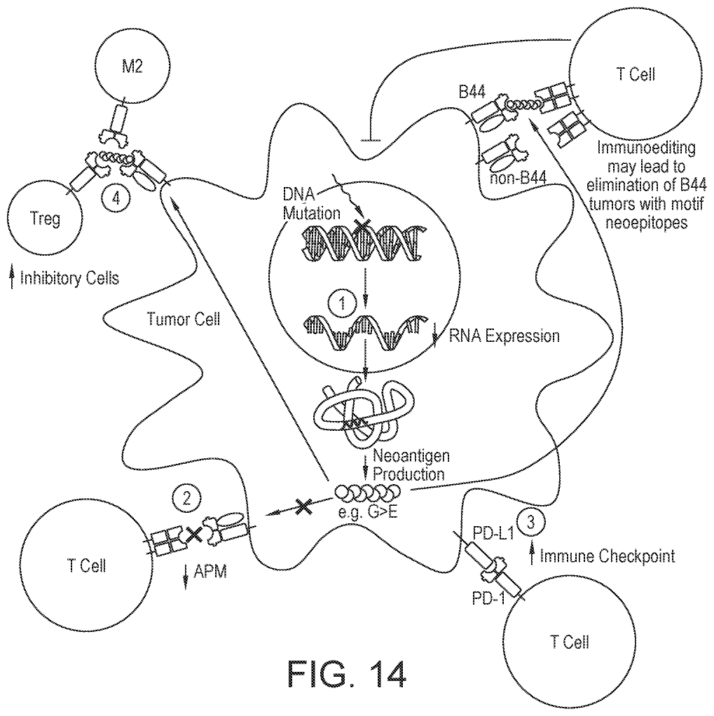

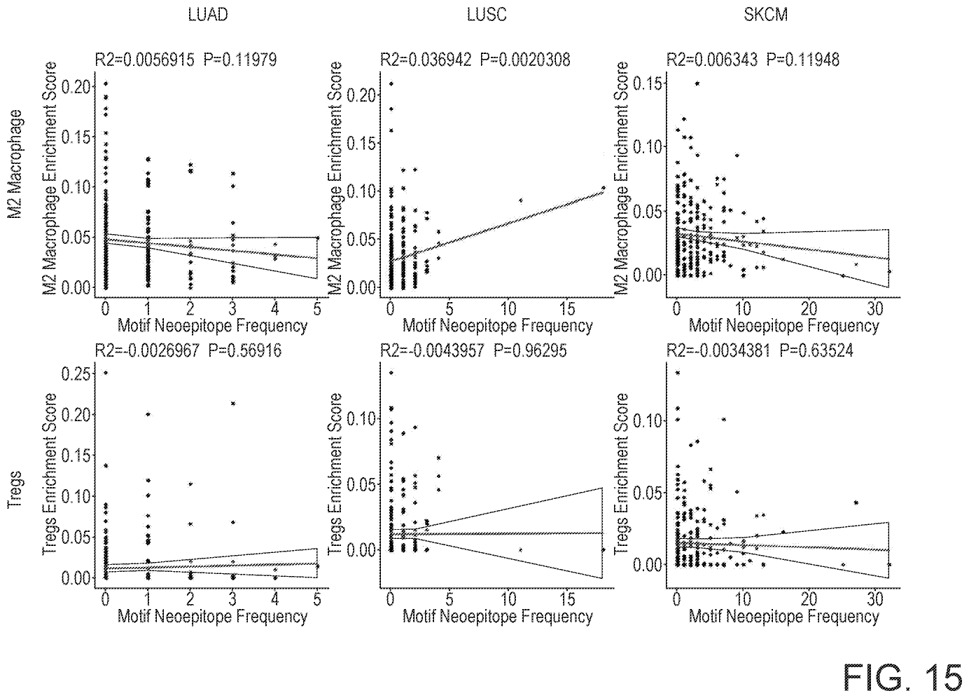

Nevertheless, tumors with favorable neoepitopes matched to host HLA alleles develop despite immune surveillance and editing, suggesting a role for immune escape mechanisms that circumvent selective pressure. These tumors may depend on escape mechanisms such as deactivation of antigen presentation machinery (APM),(24,25) modification of the tumor microenvironment (TME),(26) and induction of immune checkpoints to evade immune surveillance (FIG. 14). Thus, it is conceivable that tumors expressing both favorable neoepitopes and immune checkpoints would demonstrate robust response to ICB.

To investigate evidence of immune editing and escape in tumors with favorable neoepitopes, we evaluated data from TCGA, assessing B44 tumors with motif-matched neoepitopes in lung adenocarcinoma (LUAD), lung squamous cell carcinoma (LUSC), and cutaneous melanoma (SKCM). These tumor types were selected based on our prior data demonstrating improved survival in ICB-treated NSCLC and melanoma patients with well-defined neoepitopes.(14) Additionally, we investigated the effect of B44 motif-matched neoepitopes in conjunction with PD-L1 expression on clinical outcomes in NSCLC patients treated with single agent anti-PD-1 therapy.

Materials and Methods

Processing of Genomic Data and Neoepitope Prediction

BAM files for chromosome six from blood derived normal samples were downloaded using the Genomic Data Commons (GDC) BAM slicing application. OptiType v1.3.1 software was used to perform HLA typing.(27) Supertype was determined by 2008 criteria.(9) Variant Call Files (VCFs), Fragments Per Kilobase Million (FPKM), and clinical data were downloaded with the GDC Data Transfer Tool. VCFs characterizing somatic mutations were annotated with SnpEff v4.3 and Ensembl Variant Effect Predictor (VEP) v99.(28,29) VCFs were additionally annotated for gene expression using VCF Annotation Tools (VAtools) v4.0. Neoepitopes for HLA-B that are nine amino acids (AAs) in length and their half maximal inhibitory concentration (IC₅₀) values were predicted from nonsynonymous mutations with Personalized Variant Antigens by Cancer Sequencing (pVAC-Seq) software v1.5.1 using NetMHCpan 4.0 algorithm. Neoepitopes were filtered with predicted IC₅₀≤500 nM and mutant score less than wildtype score.(30,31)

Definitions of Radical Substitutions and Motif/Non-Motif Neoepitopes

Aspartic Acid (D) and glutamic acid (E) were considered negatively charged AAs and histidine (H), lysine (K), and arginine (R) were considered positively charged, based on side chain pKₐ values.(32) All other AAs were considered uncharged. Radical substitutions were defined as somatic mutations producing charged AAs from oppositely charged or uncharged wildtype AAs. Motif neoepitopes for B44 were defined as nonamers with radical negative substitutions (D/E) in the anchor (2^{nd}) position and well-defined supertype C-terminus (FWYLIVM; SEQ ID NO: 4).(33) Non-motif neoepitopes were defined as nonamers without radical negative substitutions in the anchor position or those with motif-matched anchor residues but without B44 supertype C-terminus residues.

Processing of Antigen Presentation Machinery, Immune Cell Infiltrate, Cytokine Levels, and Immune Checkpoint Gene Data

The TCGAbiolinks package available in R was used for analysis of APM genes and immune checkpoint gene expression.(34,35) Masked somatic Mutation Annotation Format (MAF) files that are processed to remove low quality and potential germline variants were downloaded. Genes involved in HLA-B antigen processing and presentation (HLA-B, 82M, TAP1, TAP2, TAPBP, PSM85, PSM86, PSM87, PSM88, PSM89, PSM810, ERAP1, ERAP2, PDIA3, CANX, CALR) were selected for analysis of somatic mutations.(36) To account for high polymorphism, HLA somatic mutations were obtained using Polysolver software from Castro et al., 2019,(24) as MAF files with mutation calls against a reference genome are inaccurate. Cell subset enumeration was performed using the xCell package in R, which uses a gene signature-based method to infer cell types.(37) Enrichment of immune inhibitory M2 macrophages and T regulatory cells (Tregs) was assessed in addition to ten other immune cell types (B cell, memory B cell, CD4 T cell, CD4 memory T cell, CD8 T cell, CD8 memory T cell, NK cell, dendritic cell, monocytes, M1 macrophages). Cytokines associated with recruitment of Tregs (CCL1, CCL17, CCL22, CCL28)(38) and M2 macrophage polarization (IL-4, IL-6, IL-10, IL-13)(39) were evaluated. PD-L1, CTLA-4, LAG-3, TIM-3, TIGIT, VISTA, 87-H3, and BTLA immune checkpoint genes were selected for evaluation of expression as these have been the most studied and have therapeutic approval and/or clinical trials underway.(40-43) RNA HTSeq counts for cytokine and immune checkpoint genes were downloaded and normalized with the integrated EDASeq package and log 2 transformed.(44) PD-L1 protein abundance was evaluated with level 4 reverse-phase protein array (RPPA) data obtained from The Cancer Proteome Atlas (TCPA).(45)

Patient Cohorts and Sample Selection

The sample containing the greatest number of VEP annotations was selected for patients with more than one VCF. Only patients that had all data types available (blood derived normal BAM files, VCFs, FPKM, and clinical information) were included in the final analysis. Methods regarding patient selection, response assessment, and sample processing of the NSCLC cohort treated on clinical trials with single-agent pembrolizumab at the University of California, Los Angeles (UCLA) have been previously reported.(14) Long term survival data with accompanying whole exome sequencing (WES) and PD-L1 IHC were available for 35 patients. An additional cohort of 22 NSCLC samples treated with single-agent pembrolizumab with availability of WES and PD-L1 IHC from Memorial Sloan Kettering Cancer Center (MSKCC) was used for survival analysis and described elsewhere.(46) Data for a third cohort from Sidney Kimmel Comprehensive Cancer Center (SKCCC) of 55 NSCLC patients treated with single-agent anti-PD-1 was also obtained for analysis.(47) High tumor proportion score (TPS) for PD-L1 was defined as 50%. TMB-H was defined as ≥10 mutations/megabase.

Statistical Methods

Univariable and multivariable logistic regression models for B44 status were constructed and summarized with odds ratios (ORs). Logistic regression models evaluated for association between presence of B44 and fraction of radical substitutions producing negatively charged AAs, as well as patient characteristics (age, gender, stage, race, TMB).

Presence of gene expression (FPKM value>0) of motif and non-motif neoepitopes in B44 patients was assessed with chi-square tests, and degree of expression was evaluated with a clustered Wilcoxon rank-sum test using the Rosner-Glynn-Lee (RGL) method to account for repeated measures. Using normalized RNA counts, average expression of genes harboring motif and non-motif neoepitopes was calculated across all samples in the B44 cohort. Fold change in expression of individual genes harboring neoepitopes was determined as a comparison to average expression of that particular gene throughout the cohort by tumor type. Fold change in expression of motif and non-motif neoepitopes was compared by Wilcoxon rank-sum test with RGL correction. Of note, normalization of RNA counts removes genes with zero value in all samples, thus predicted neoepitopes without expression in any sample were discarded. Protein analysis through evolutionary relationships (PANTHER) gene ontology was used to assess protein class overrepresentation, comparing genes harboring motif neoepitopes to a human reference genome. The comparison was statistically analyzed using Fisher's test with correction for multiple hypothesis testing by false discovery rate (FDR).

Presence of mutations in APM genes was compared with presence of motif neoepitopes among B44 patients using chi-square tests. Multivariate logistic regression was performed to measure influence of TMB-H on presence of APM gene mutations. To assess loss of heterozygosity (LOH) of the HLA-B44 allele, HLA-B type between tumor and matched normal sample was assessed, with LOH defined as tumor demonstrating homozygosity of the non-B44 allele and normal demonstrating heterozygosity (B44/non-B44 HLA-B alleles). LOH among samples was compared with chi-square test.

Immune cell enrichment, cytokine levels, and immune checkpoint gene expression were assessed using Wilcoxon tests in B44 patients with and without motif neoepitopes. Frequency of motif neoepitopes per patient was compared to immune inhibitory cell enrichment scores and immune checkpoint gene expression using linear regression and Pearson's coefficient test. Correlation between PD-L1 RNA counts and RPPA was evaluated with linear regression tests.

Overall survival (OS) and progression-free survival (PFS) were computed using the Kaplan-Meier method and compared between groups using non-parametric log-rank tests. Hazard ratios (HRs) were estimated using proportional hazards. All analyses other than logistic regression models for B44 were restricted to patients harboring at least one HLA-B44 allele. P-values<0.05 were considered statistically significant. Statistical analyses were performed in R v4.0 and SAS v9.4 (Cary, NC).

Code availability is through: github.com/FRED-2/OptiType, htslib.org, pcingola.github.io/SnpEff, ensembl.org, vatools.readthedocs.io, pvacseq.readthedocs.io, bioconductor.org/packages/release/bioc/html/TCGAbiolinks.html, github.com/dviraran/xCell.

Results

Presence of B44 Associates with Degree of Radical Negative Substitutions in LUAD

For patients that had availability of all data types (LUAD=511, LUSC=482, SKCM=466), median age was 66, 68, and 58 in LUAD, LUSC, and SKCM cohorts, respectively; and 53.8%, 25.9%, and 38.2% were female. The majority of patients were white (75.3%, 70.1%, 95.1%) and had early stage disease (stage I, II, Ill). At least one B44 allele was present in 46.8% of LUAD, 47.3% of LUSC, and 48.7% of SKCM patients (Table

TABLE 5
Patient demographics by tumor type and HLA-B44 status

| Histology | LUAD | | | |
|---|---|---|---|---|
| Group | | Total | B44 | Non-B44 |

| | | | | | |
|---|---|---|---|---|---|
| Patient Number | 511 | 239 | (46.8) | 272 | (53.2) |

| | | | | | | |
|---|---|---|---|---|---|---|
| Age | 66 | (33-88) | 65 | (33-88) | 67 | (39-87) |
| Female | 275 | (53.8) | 116 | (48.6) | 159 | (58.5) |
| Male | 236 | (46.2) | 123 | (51.4) | 113 | (41.5) |
| White | 385 | (75.3) | 180 | (75.3) | 205 | (75.4) |
| Black | 52 | (10.2) | 19 | (7.9) | 33 | (12.1) |
| Asian | 7 | (1.4) | 2 | (0.8) | 5 | (1.8) |
| Other/NR Race | 67 | (13.1) | 38 | (15.9) | 29 | (10.7) |
| Stage I | 273 | (53.4) | 117 | (49.0) | 156 | (57.4) |
| Stage II | 120 | (23.5) | 70 | (29.3) | 50 | (18.4) |
| Stage III | 84 | (16.4) | 35 | (14.7) | 49 | (18.0) |
| Stage IV | 26 | (5.1) | 15 | (6.3) | 11 | (4.0) |
| Stage NR | 8 | (1.6) | 2 | (0.9) | 6 | (2.2) |
| TMB | 5.4 | (0.1-34.8) | 5.8 | (0.1-34.8) | 5.1 | (0.1-27.6) |

| LUSC | | | | | | SKCM | | | | | |
|---|---|---|---|---|---|---|---|---|---|---|---|
| Total | | B44 | | Non-B44 | | Total | | B44 | | Non-B44 | |
| 482 | | 228 | (47.3) | 254 | (52.7) | 466 | | 227 | (48.7) | 239 | (51.3) |
| 68 | (39-90) | 68 | (40-90) | 68 | (39-84) | 58 | (15-90) | 56 | (18-90) | 61 | (15-90) |
| 125 | (25.9) | 63 | (27.7) | 62 | (24.4) | 178 | (38.2) | 88 | (38.8) | 90 | (37.7) |
| 357 | (74.1) | 165 | (72.3) | 192 | (75.6) | 288 | (61.8) | 139 | (61.2) | 149 | (62.3) |
| 338 | (70.1) | 162 | (71.1) | 176 | (69.3) | 443 | (95.1) | 217 | (95.6) | 226 | (94.6) |
| 29 | (6.0) | 13 | (5.7) | 16 | (6.3) | 1 | (0.2) | NA | | 1 | (0.4) |
| 8 | (1.7) | 4 | (1.8) | 4 | (1.6) | 12 | (2.6) | 5 | (2.2) | 7 | (2.9) |
| 107 | (22.2) | 49 | (21.5) | 58 | (22.8) | 10 | (2.5) | NA | | 5 | (2.1) |
| 111 | (48.7) | 111 | (48.7) | 122 | (48.0) | 76 | (16.3) | 34 | (15.0) | 42 | (17.6) |
| 71 | (31.1) | 71 | (31.1) | 86 | (33.9) | 140 | (30.0) | 69 | (30.4) | 71 | (29.7) |
| 81 | (16.9) | 35 | (15.4) | 46 | (18.1) | 168 | (36.1) | 89 | (39.2) | 79 | (33.1) |
| 7 | (3.1) | 7 | (3.1) | NA | | 23 | (4.9) | 14 | (6.2) | 9 | (3.8) |
| 4 | (1.8) | 4 | (1.8) | NA | | 59 | (12.7) | 21 | (9.3) | 38 | (15.9) |
| 5.0 | (0.03-46.8) | 5.2 | (0.03-46.8) | 4.8 | (0.1-30.6) | 11.8 | (0.2-230) | 10.6 | (0.2-111.9) | 12.9 | (0.2-230) |

Patient number represents total samples in each group with percentage of total in parentheses. Age and TMB displayed as median value and range in parentheses. Sex, race and stage are shown as count and percentage of total in parentheses. LUAD—lung adenocarcinoma, LUSC—lung squamous cell carcinoma, SKCM—cutaneous melanoma, B44—human leukocyte antigen B44 supertype, NA—not applicable, NR—not reported, TMB—tumor mutational burden.

To evaluate for evidence of immunoediting, we assessed presence of B44 in relation to mutational landscape, hypothesizing that tumors in individuals with B44 are less likely to harbor mutations resulting in negatively charged amino acid substitutions, as these substitutions can produce motif neoepitopes with favorable binding and presentation. In LUAD, presence of B44 showed an inverse association with fraction of negatively-charged radical substitutions, with univariable odds ratio (OR) of 0.25, p=0.03 and similar multivariable OR of 0.24, p=0.04, suggesting decreased odds of LUAD tumors having B44 with increasing proportion of radical substitutions that are negatively charged (Table 4A). A similar trend was observed in LUSC tumors (multivariable OR 0.37, p=0.29), but not SKCM (Table 4B-C).

Table 4A-4C. Univariable and Multivariable Logistic Regression Models for Presence of HLA-B44 by Tumor Type.

ORs for presence of B44 (true/false). Variables age, TMB, and proportion of new charged amino acids that are negative are reported as unit increases. Convergence reported when unable to accurately calculate an odds ratio due to lack of samples. LUAD—lung adenocarcinoma, LUSC—lung squamous cell carcinoma, SKCM—cutaneous melanoma, B44—human leukocyte antigen B44 supertype, OR—odds ratio, CI—confidence interval, NA—not applicable, TMB—tumor mutational burden, AAs—amino acids.

TABLE 4A
LUAD logistic regression models for B44, n = 511

| | LUAD B44 (True/False) Odds Ratios | | | |
|---|---|---|---|---|
| | Univariable | | Multivariable | |
| Variable | OR (95% CI) | p-value | OR (95% CI) | p-value |
| Age | 0.99 (0.98-1.01) | 0.42 | 0.99 (0.97-1.01) | 0.40 |
| Male vs Female | 1.49 (1.05-2.12) | 0.03 | 1.35 (0.93-1.96) | 0.12 |
| Race | | | | |
| African American vs White | 0.66 (0.36-1.19) | 0.17 | 0.57 (0.31-1.07) | 0.08 |
| Asian vs White | 0.46 (0.09-2.38) | 0.35 | 0.37 (0.07-2.01) | 0.25 |
| Other/Unknown vs White | 1.49 (0.88-2.52) | 0.13 | 1.20 (0.64-2.26) | 0.57 |
| Stage | | | | |
| Stage I vs IV | 0.55 (0.24-1.24) | 0.15 | 0.52 (0.22-1.23) | 0.14 |
| Stage II vs IV | 1.03 (0.44-2.42) | 0.95 | 0.97 (0.39-2.43) | 0.95 |
| Stage III vs IV | 0.52 (0.22-1.28) | 0.16 | 0.51 (0.20-1.32) | 0.16 |
| Stage Unknown vs IV | 0.24 (0.04-1.45) | 0.12 | 0.23 (0.04-1.43) | 0.12 |
| TMB | 1.03 (0.99-1.06) | 0.12 | 1.02 (0.98-1.05) | 0.36 |
| Proportion Negative | 0.25 (0.07-0.89) | 0.03 | 0.24 (0.06-0.94) | 0.04 |
| of Charged AAs | | | | |

TABLE 4B
LUSC logistic regression models for B44, n = 482

| | LUSC B44 (True/False) Odds Ratios | | | |
|---|---|---|---|---|
| | Univariable | | Multivariable | |
| Variable | OR (95% CI) | p-value | OR (95% CI) | p-value |
| Age | 1.00 (0.98-1.02) | 0.80 | 1.00 (0.98-1.02) | 0.78 |
| Male vs Female | 0.85 (0.56-1.27) | 0.42 | 0.84 (0.55-1.27) | 0.40 |
| Race | | | | |
| African American vs White | 0.88 (0.41-1.89) | 0.75 | 0.92 (0.42-1.99) | 0.83 |
| Asian vs White | 1.09 (0.27-4.42) | 0.91 | 0.97 (0.21-4.50) | 0.97 |
| Other/Unknown vs White | 0.92 (0.59-1.42) | 0.70 | 0.87 (0.54-1.39) | 0.55 |
| Stage | | | | |
| Stage I vs IV | Convergence | NA | Convergence | NA |
| Stage II vs IV | Convergence | NA | Convergence | NA |
| Stage III vs IV | Convergence | NA | Convergence | NA |
| Stage Unknown vs IV | Convergence | NA | Convergence | NA |
| TMB | 1.02 (0.98-1.07) | 0.31 | 1.02 (0.98-1.07) | 0.35 |
| Proportion Negative of | 0.34 (0.06-2.10) | 0.25 | 0.37 (0.06-2.36) | 0.29 |
| Charged AAs | | | | |

TABLE 4C
SKCM logistic regression models for B44, n = 466

| | SKCM B44 (True/False) Odds Ratios | | | | |
|---|---|---|---|---|---|
| | Univariable | | | Multivariable | |
| Variable | OR (95% CI) | | p-value | OR (95% CI) | p-value |
| Age | 0.99 | (0.97-1.00) | 0.02 | 0.98 (0.97-1.00) | 0.01 |
| Male vs Female | 0.95 | (0.66-1.39) | 0.81 | 0.95 (0.64-1.40) | 0.78 |
| Race | | | | | |
| African American vs White | Convergence | | NA | Convergence | NA |
| Asian vs White | 0.74 | (0.23-2.38) | 0.62 | 0.52 (0.14-1.94) | 0.33 |
| Other/Unknown vs White | 1.04 | (0.30-3.65) | 0.95 | 1.11 (0.31-4.06) | 0.87 |
| Stage | | | | | |
| Stage I vs IV | 0.47 | (0.18-1.21) | 0.12 | 0.52 (0.19-1.37) | 0.18 |
| Stage II vs IV | 0.63 | (0.25-1.54) | 0.31 | 0.82 (0.32-2.10) | 0.68 |
| Stage III vs IV | 0.72 | (0.30-1.76) | 0.48 | 0.89 (0.35-2.24) | 0.80 |
| Stage Unknown vs IV | 0.40 | (0.15-1.10) | 0.08 | 0.43 (0.15-1.26) | 0.12 |
| TMB | 0.99 | (0.98-1.00) | 0.15 | 0.99 (0.98-1.01) | 0.28 |
| Proportion Negative of | 2.87 | (0.70-11.76) | 0.14 | 2.12 (0.50-9.09) | 0.31 |
| Charged AAs | | | | | |

For unclear reasons, younger age (OR 0.99, p=0.03; driven entirely by SKCM) and stage IV disease (ORs 0.37-0.53, p=0.01-0.04) had increased odds of possessing a B44 allele (Table 6). Additionally, there was a trend towards African American (OR 0.69, p=0.13) and Asian (OR 0.63, p=0.27) patients being less likely to harbor a B44 allele compared to white patients.

TABLE 6
Univariable and multivariable logistic regression
models for presence of HLA-B44

| | B44 (True/False) Logistic Regression | | | | | |
|---|---|---|---|---|---|---|
| | Univariable-OR | | p- | Multivariable-OR | | p- |
| Variable | (95%-CI) | | value | (95%-CI) | | value |
| Age | 0.99 | 0.98-0.99 | 0.03 | 0.99 | 0.98-1.00 | 0.03 |
| Male vs Female | 1.10 | 0.89-1.35 | 0.40 | 1.05 | 0.84-1.32 | 0.66 |
| African American | 0.70 | 0.44-1.10 | 0.12 | 0.69 | 0.43-1.11 | 0.13 |
| vs White | | | | | | |
| Asian vs White | 0.75 | 0.34-1.62 | 0.46 | 0.63 | 0.28-1.43 | 0.27 |
| Other/Unknown | 1.09 | 0.80-1.48 | 0.60 | 0.97 | 0.69-1.39 | 0.88 |
| vs White | | | | | | |
| Stage 1 vs 4 | 0.45 | 0.25-0.79 | 0.01 | 0.45 | 0.25-0.82 | 0.01 |
| Stage 1 vs 4 | 0.56 | 0.32-1.01 | 0.05 | 0.59 | 0.32-1.07 | 0.08 |
| Stage 1 vs 4 | 0.51 | 0.28-0.91 | 0.02 | 0.53 | 0.29-0.98 | 0.04 |
| Stage Unknown vs 4 | 0.38 | 0.18-0.80 | 0.01 | 0.37 | 0.17-0.80 | 0.01 |
| TMB | 1.00 | 0.99-1.01 | 0.65 | 1.00 | 0.99-1.01 | 0.58 |
| Proportion Negative | 0.65 | 0.29-1.46 | 0.30 | 0.58 | 0.24-1.43 | 0.24 |
| Charged AAs | | | | | | |

ORs for presence of B44 (true/false). Variables age, TMB, and proportion of new charged amino acids that are negative are reported as unit increases. Total n = 1459, LUAD n = 511, LUSC n = 482, SKCM n = 466. LUAD—lung adenocarcinoma, LUSC—lung squamous cell carcinoma, SKCM—cutaneous melanoma, B44—human leukocyte antigen B44 supertype, OR—odds ratio, CI—confidence interval, TMB—tumor mutational burden, AAs—amino acids.

These results are consistent with immunoediting in LUAD, and to a lesser extent in LUSC, which selects against mutations that putatively produce B44 motif neoepitopes. However, immunoediting does not appear universal across tumor types given lack of evidence in SKCM, suggesting that additional mechanisms are employed by tumors to escape immune surveillance in the setting of immunogenic neoepitopes.

Motif Neoepitopes are Expressed at Lower Levels than Non-Motif Neoepitopes in SKCM

Next, we hypothesized that gene expression of motif neoepitopes may be decreased compared to non-motif neoepitopes as a mechanism of immune escape in B44 patients. Assessing for evidence of any gene expression, we found that motif neoepitopes were less likely to have detectable expression than non-motif, reaching statistical significance in LUAD (χ2=8.56, p=0.003) and SKCM (χ2=59.7, p<0.001), but not LUSC (χ2=0.24, p=0.62) (FIG. 9A and Table 6). When measuring degree of RNA expression, a trend towards decreased expression of motif neoepitopes was seen across lung histologies, with a strong association observed in SKCM (W=−9.6, p<0.001) (FIG. 9B).

TABLE 7
Motif and non-motif neoepitope expression characteristics

| Histology | LUAD | LUSC | SKCM |
|---|---|---|---|
| Total patients | 511 | 482 | 466 |
| B44 patients | 239 | 228 | 227 |
| Patients with motif | 100 | 95 | 156 |
| Patients with expressed motif | 89 | 85 | 148 |
| Total motif neoepitopes | 164 | 176 | 690 |
| Expressed motif necepitopes | 135 | 154 | 529 |
| Non-expressed motif neoepitopes | 29 | 22 | 161 |
| Proportion expressed motifs | 82.30% | 87.50% | 76.67% |
| Patients with non-motif | 222 | 221 | 218 |
| Patients with expressed non-motif | 220 | 220 | 214 |
| Total non-motif neoepitopes | 2590 | 2157 | 4638 |
| Expressed non-motif neoepitopes | 2328 | 1920 | 4064 |
| Non-expressed non-motif neoepitopes | 262 | 237 | 574 |
| Proportion expressed non-motifs | 89.90% | 90.97% | 87.62% |
| Motif Expression Log2 FPKM Median (IQR) | −0.05 (−3.47-2.41) | 0.44 (−2.44-2.41) | −2.87 (−5.8-0.28) |
| Non-motif Expression Log2 FPKM Median (IQR) | 0.62 (−2.99-2.71) | 0.63 (−2.83-2.61) | 0.23 (−3.59-2.66) |

To account for physiological levels of gene transcription, we then compared fold change in expression of motif and non-motif neoepitopes, considering mean expression of genes producing neoepitopes across all samples in the cohorts as reference. We saw that expression of neoepitopes was less than cohort means of the corresponding genes, and that SKCM continued to show significantly decreased expression of motif as compared to non-motif neoepitopes (W=−4.81, p<0.001) (FIG. 9C). Taken together, these results suggest that SKCM tumors, a histology for which immunoediting was not seen, are compelled to decrease expression of motif neoepitopes, which could play a role in immune evasion.

Additionally, protein class representation analysis was performed to determine if motif neoepitope-producing mutations occurred in genes with particular features. We observed that several protein classes were overrepresented by genes harboring motif neoepitopes across all tumor types, particularly transmembrane proteins Table 8).

TABLE 8
Protein class representation for motif neoepitope containing genes

| | | | | | |
|---|---|---|---|---|---|
| Protein Class | Expected | Observed | Enrichment | P-value | FDR |

LUAD

| | | | | | |
|---|---|---|---|---|---|
| Transmembrane Signal | 8.33 | 29 | 3.48 | 5.39E−09 | 1.04E−06 |
| Receptor | | | | | |

LUSC

| | | | | | |
|---|---|---|---|---|---|
| Transmembrane Signal | 9.13 | 22 | 2.41 | 2.13E−04 | 0.041 |
| Receptor | | | | | |
| Cadherin | 0.94 | 6 | 6.39 | 4.60E−04 | 0.044 |

SKCM

| | | | | | |
|---|---|---|---|---|---|
| Voltage-Gated Ion Channel | 2.64 | 15 | 5.69 | 2.85E−07 | 9.16E−06 |
| Extracellular Matrix | 2.17 | 12 | 5.54 | 5.65E−06 | 1.36E−04 |
| Structural Protein | | | | | |
| Cadherin | 3.31 | 14 | 4.23 | 1.58E−05 | 3.39E−04 |
| Primary Active Transporter | 6.18 | 15 | 2.43 | 2.14E−03 | 2.43E−02 |
| G-protein Coupled Receptor | 11.39 | 24 | 2.11 | 1.35E−03 | 1.73E−02 |
| RNA Metabolism Protein | 20 | 8 | 0.4 | 3.52E−03 | 3.58E−02 |
| C2H2 Zinc Finger | 13.4 | 3 | 0.22 | 1.58E−03 | 1.91E−02 |
| Transcription Factor | | | | | |

Gene ontology protein class over and under representation for genes with motif neoepitope producing mutations. Distribution of motif neoepitope containing genes was compared to distribution of genes in human reference genome using Fisher's test with false discovery rate (FDR) correcting for multiple hypothesis testing.

LUSC and SKCM Tumors with Motif Neoepitopes Demonstrate Mutations in Antigen Presentation Machinery Genes:

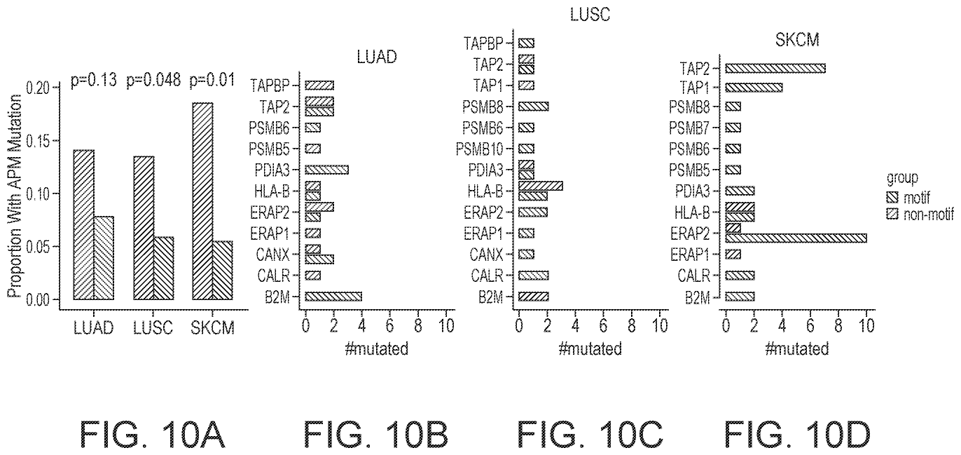

In addition to loss of immunogenic mutations, immune escape can occur through deactivation of APM, resulting in inability to present neoantigens. To investigate if loss of antigen presentation may serve as an immune escape mechanism in the presence of favorable neoepitopes, we evaluated the relationship between somatic mutations in APM and presence of motif neoepitopes in B44 patients, predicting that tumors with motif neoepitopes are more likely to have mutations in APM genes. We found that LUSC (χ2=3.89, p=0.048) and SKCM (χ2=6.59, p=0.01) tumors with motif neoepitopes were more likely to harbor somatic mutations in at least one APM gene than those without motif neoepitopes (FIG. 10A). In LUAD, a larger proportion of tumors with motif neoepitopes had APM mutations, however this did not reach statistical significance (χ2=2.3, p=0.13). When accounting for TMB-H status, the association of APM mutations and motif neoepitopes was similar compared to when not accounting for TMB-H in LUSC (p=0.06), but was less robust in SKCM (p=0.21) and LUAD (p=0.78).

Genes harboring motif neoepitopes did not demonstrate co-occurrence of somatic mutations with APM genes in LUAD and LUSC. However, 3 of 697 genes with motif neoepitopes in SKCM were classified as APM genes (PDIA3, PSMB6, PSMB8), although APM protein class was not over or underrepresented when compared to a human reference genome (Table 9). Additionally, LOH of HLA-B44 was infrequently seen among tumor types, and difference in LOH between tumors with and without motif neoepitopes was not significant (Table 10). Mutated genes within each histology are summarized in FIG. 10B-D.

TABLE 9
APM genes with motif neoepitope producing mutations in SKCM

| ID | Reference | Variant | Mutation | Gene Name |
|---|---|---|---|---|
| TCGA-EE-A2MR | G | A | G/E | PDIA3 |
| TCGA-EE-A2GR | C | T | G/E | PSMB8 |
| TCGA-FS-A4F0 | G | A | G/E | PSMB6 |

Of 697 genes harboring motif neoepitope producing mutations, 3 were classified as antigen presentation machinery genes (PDIA3, PSMB6, PSMB8). Comparing to PANTHER human reference genome gene ontology, antigen processing and presentation genes were not over or underrepresented in SKCM (expected 7, observed 3, enrichment 0.42, p-value with fisher's exact test). APM genes with motif neoepitope producing mutations in LUAD and LUSC were not observed. APM genes were not underrepresented in LUAD (expected 2, observed 0, enrichment 0, p-value 0.49) or LUSC (expected 2, observed 0, enrichment 0, p-value 0.50).

TABLE 10
Loss of heterozygosity of HLA-B44 gene

| | LUAD | | LUSC | | SKCM | |
|---|---|---|---|---|---|---|
| | Motif | Motif | Motif | Motif | Motif | Motif |
| | Present | Absent | resent | Absent | Present | Absent |
| LOH Present | 2 | 0 | 1 | 3 | 4 | 4 |
| LOH Absent | 99 | 139 | 94 | 130 | 152 | 67 |

| | | |
|---|---|---|
| χ² = 0.89, df = | χ² = 0.03, df = | χ² = 0.60, df = |
| 1, p = 0.34 | 1, p = 0.86 | 1, p = 0.44 |

Loss of heterozygosity (LOH) was determined by comparing HLA-B alleles between tumor and matched normal samples. LOH was defined as loss of B44 allele in tumor sample with tumor demonstrating homozygosity for non-B44 allele. B44 LOH for patients with motif neoepitope present and motif neoepitope absent was compared using chi-squared test. No differences in loss of heterozygosity of HLA-B44 between motif present and motif absent were seen in LUAD, LUSC, and SKCM.

Immune Inhibitory Cells Associate with Presence of Favorable Neoepitopes in LUSC:

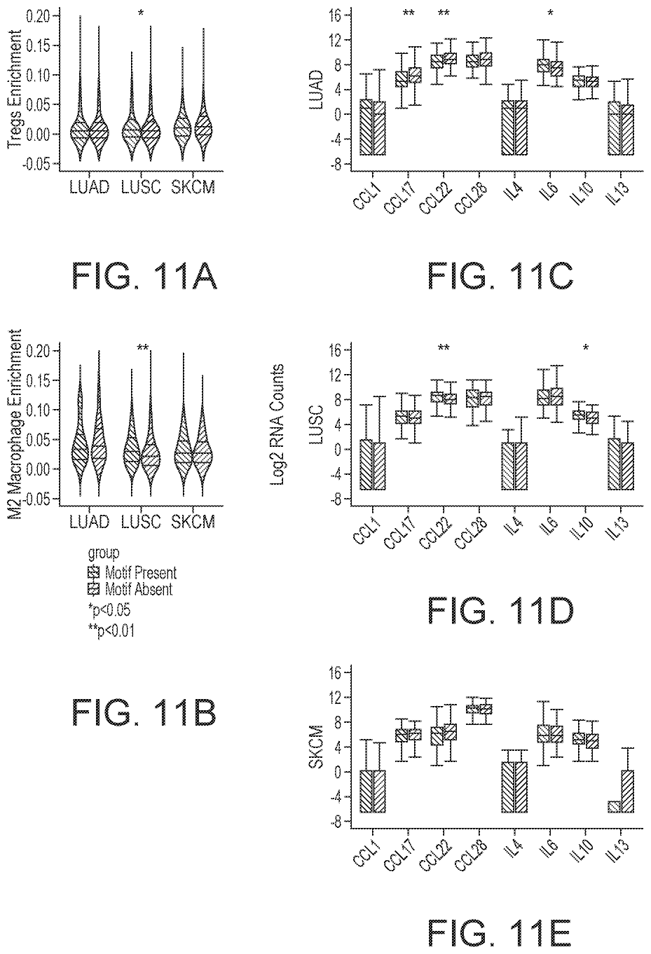

It is recognized that intratumoral immune cells can both promote or inhibit cancer development. Accordingly, we queried the relationship between immune inhibitory cells and presence of motif neoepitopes in B44 patients to infer if features within the TME could promote immune escape in the setting of immunogenic stimuli. Both immune inhibitory Tregs (p=0.04, p=0.14 when corrected for multiple hypothesis testing) and M2 macrophages (p=0.01, p=0.03 when corrected for multiple hypothesis testing) were enriched in LUSC samples with motif neoepitopes but not in LUAD or SKCM (FIG. 11A-B). These findings suggest that LUSC tumors with favorable neoepitopes may upregulate immune inhibitory cells to avoid destruction. Frequency of motif neoepitope per patient did not demonstrate meaningful correlation with M2 macrophage or Tregs enrichment among all histologies (FIG. 15).

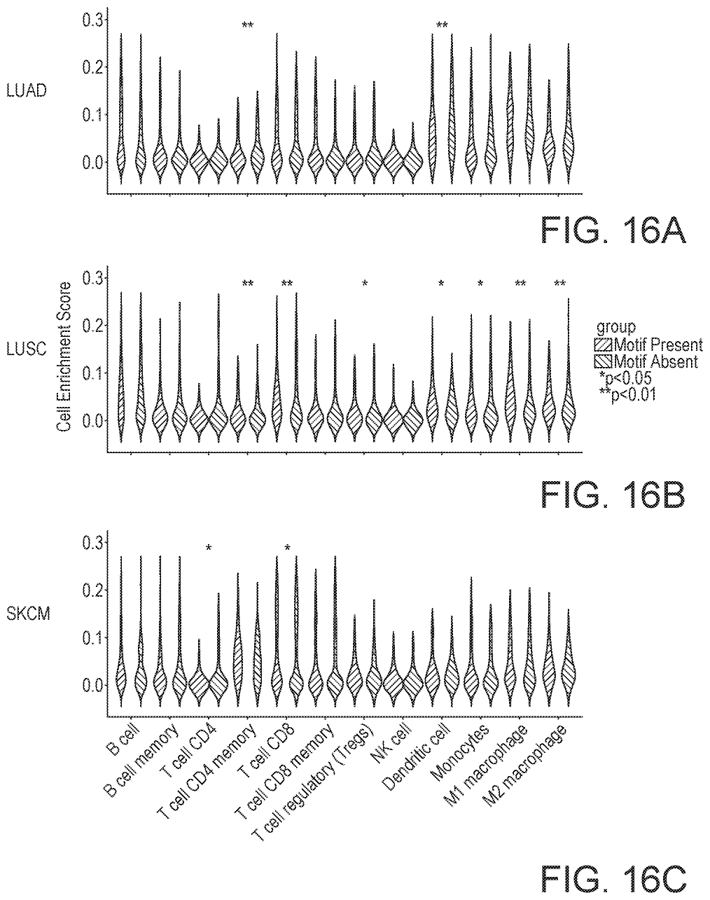

To further investigate, we assessed levels of cytokines associated with recruitment of Tregs and polarization of M2 macrophages. We found greater expression of CCL22 and IL-10 in LUSC tumors with motif neoepitopes, which have been implicated in Treg recruitment and M2 macrophage polarization, respectively (FIG. 11D). While IL-6 expression was increased in LUAD tumors with motif neoepitopes, CCL17 and CCL22 showed reduced expression and there were no differences in cytokine levels in SKCM tumors (FIGS. 11C and 11E). As immune inhibitory cells occur in the context of many cell types, we additionally assessed enrichment of ten immune stimulatory cells (FIG. 16). Overall, LUSC exhibited a greater degree of immune cell enrichment in the setting of immunogenic neoepitopes.

Induction of Immune Checkpoints in the Setting of Motif Neoepitopes:

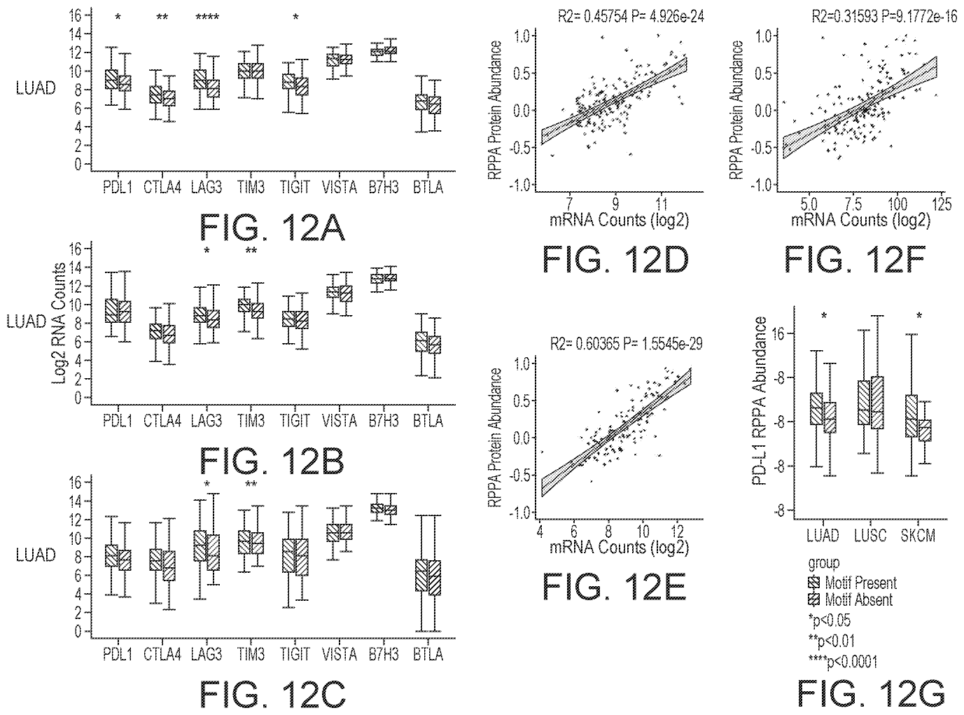

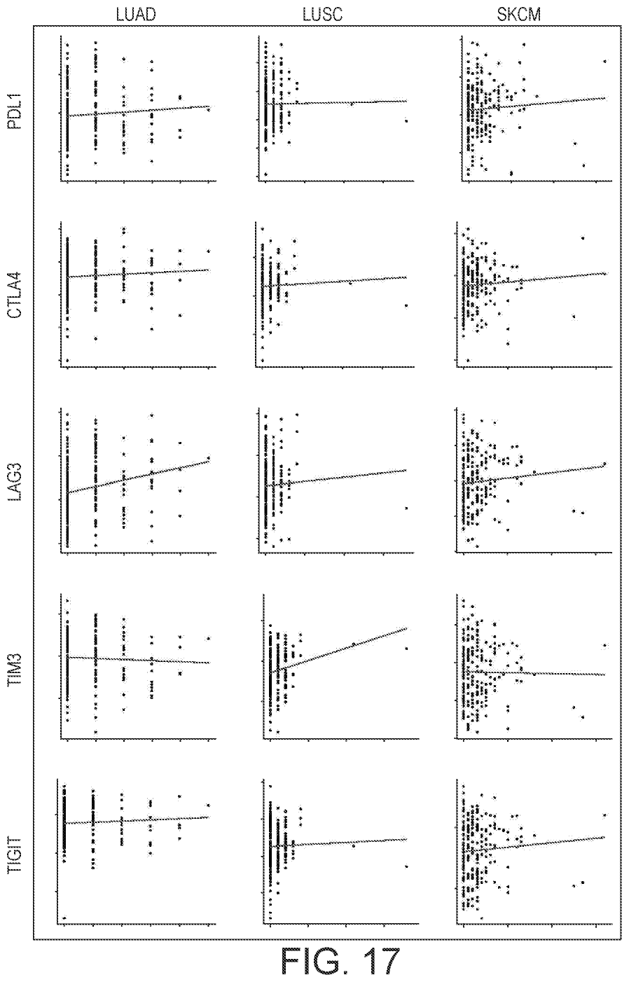

To further evaluate determinants of immune escape in the setting of immunogenic neoepitopes, we assessed the expression of multiple clinically relevant immune checkpoints in B44 patients. We postulated that tumors harboring favorable neoepitopes are more likely to experience induction of immune checkpoints, impeding surveillance and resulting in immune evasion. We found greater gene transcription of immune checkpoints among those with motif neoepitopes. This effect was strongest in LUAD, with PD-L1, CTLA-4, LAG-3, and TIGIT showing significantly increased expression in tumors with favorable neoepitopes, and to a lesser degree in LUSC (LAG-3, TIM-3), and SKCM (PD-L1, LAG-3) (FIG. 12 and Table 11). However, there were no meaningful correlations observed between increasing frequency of motif neoepitopes per patient and gene expression of select immune checkpoints (FIG. 17).

TABLE 11
Immune checkpoint gene expression in patients with
and without motif neoepitopes across tumor types

| | LUAD | LUSC | SKCM |
|---|---|---|---|
| pdl1 motif median | 8.890 | 8.807 | 8.111 |
| pdla nonmotif median | 8.476 | 9.155 | 7.679 |
| pdl1 wilcoxon p | 0.027 | 0.797 | 0.047 |
| ctla4 motif median | 7.472 | 7.066 | 7.535 |
| ctla4 nonmotif median | 6.977 | 6.615 | 6.807 |
| ctla4 wilcoxon p | 0.008 | 0.081 | 0.076 |
| lag3 motif median | 8.044 | 8.704 | 9.119 |
| lag3 nonmotif median | 8.061 | 8.299 | 8.109 |
| lag3 wilcoxon p | 0.000 | 0.050 | 0.038 |
| tigit motif median | 8.733 | 8.371 | 8.575 |
| tigit nonmotif median | 8.331 | 8.098 | 8.011 |
| tigit wilcoxon p | 0.018 | 0.186 | 0.477 |
| vista motif median | 11.293 | 11.158 | 10.553 |
| vista nonmotif median | 11.187 | 11.208 | 10.458 |
| vista wilcoxon p | 0.731 | 0.646 | 0.819 |
| tim3 motif median | 9.971 | 9.872 | 9.567 |
| tim3 nonmotif median | 10.013 | 9.160 | 9.335 |
| tim3 wilcoxon p | 0.935 | 0.006 | 0.646 |
| b7h3 motif median | 12.057 | 12.769 | 13.057 |
| b7h3 nonmotif meidan | 12.071 | 12.738 | 12.981 |
| b7h3 wilcoxon p | 0.145 | 0.588 | 0.167 |
| btla motif median | 6.728 | 6.000 | 6.418 |
| btla nonmotif median | 6.375 | 5.492 | 5.883 |
| btla wilcoxon p | 0.129 | 0.085 | 0.730 |

Immune checkpoint gene expression represented as median and derived from normalized HTSeq RNA counts that have been log 2 transformed.

Since the transcriptome and proteome relationship is complex and not directly proportional, we evaluated if PD-L1 RNA counts are correlated with protein abundance. Although transcription of PD-L1 was significantly correlated with protein levels in all three tumor types, the strength of the relationship was not particularly robust (LUAD R2=0.46, LUSC R2=0.60, SKCM R2=0.32) (FIG. 12D-F), potentially indicating presence of post-transcriptional/translational modifications. Nevertheless, similar to gene expression analysis, PD-L1 protein abundance was increased in LUAD and SKCM tumors with motif neoepitopes (FIG. 12G). Results from this analysis could indicate that distinctive tumor types rely on varying degrees of immune checkpoint induction as a mechanism of immune escape in the setting of motif neoepitopes. Additionally, particular immune checkpoints may play a larger role in immune escape within specific histologies and serve as preferential targets for immunotherapy.

Survival Associates with Presence of Motif Neoepitopes and Expression of PD-L1 in NSCLC:

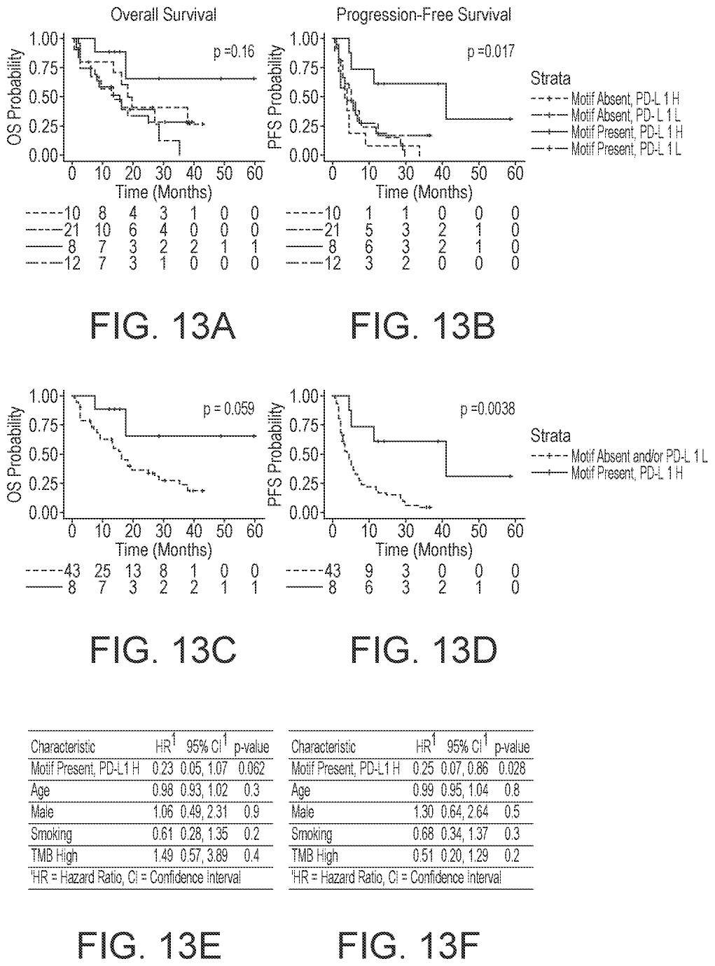

Finally, we speculated that tumors with both B44 motif-matched neoepitopes and expression of PD-L1 would show robust long-term outcomes with ICB as these tumors may be disproportionately reliant on induction of PD-L1 for immune escape. We evaluated OS and PFS in three combined cohorts of NSCLC patients treated with single-agent pembrolizumab or nivolumab in subsequent therapy. Only patients possessing at least one B44 allele and had PD-L1 IHC data available were included in the final analysis (Table 12). There was a trend towards improved long-term OS in patients with motif neoepitopes and high PD-L1 expression as compared to patients that lacked motif neoepitopes and/or did not express PD-L1 at a TPS of ≥50% (FIG. 13A), with PFS reaching statistical significance (FIG. 13B). Similar results were observed when combining survival data for motif neoepitope absent and/or PD-L1 low groups (FIGS. 13C and 13D), with median OS not reached vs 16 months (multivariable HR 0.23, p=0.06) and median PFS of 31 months vs 4.5 months (multivariable HR 0.25, p=0.028) in patients with motif neoepitopes and high PD-L1 expression as compared to those without. Multivariable analyses for survival also showed a trend towards protective effects from smoking on OS and PFS and TMB-H on PFS (FIGS. 13E and 13F).

TABLE 12
Patient demographics for NSCLC patients
treated with single-agent anti-PD-1

| | |
|---|---|
| Total Patients | B44 Patients |

| | | |
|---|---|---|
| Total | 112 | 53 |

| | | | | |
|---|---|---|---|---|
| B44 | 53 | (47.3%) | 53 | (100%) |
| Motif | 22 | (19.6%) | 22 | (41.5%) |
| PDL1 ≥ 50% | 38 | (33.9%) | 18 | (34%) |
| Age | 64 | (32-88) | 65 | (32-88) |
| Male | 61 | (54.5%) | 31 | (58.5%) |
| Smoking | 79 | (70.5%) | 37 | (69.8%) |

Shown as total patients (n=112) and patients with HLA-B44 (n=53). Final cohort is a combination of UCLA cohort (35 patients), MSKCC cohort (22 patients), SKCCC cohort (55 patients). Total patients determined after accounting for overlap in UCLA and MSKCC cohorts. Only patients with whole exome sequencing and PD-L1 IHC data were included.

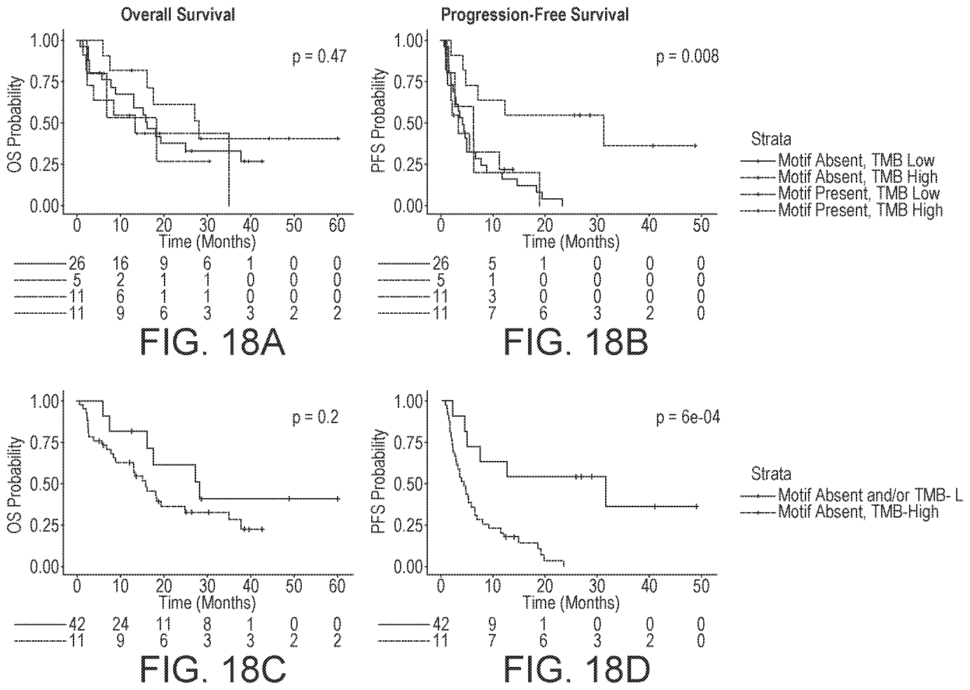

Analogous PFS results were observed when assessing survival based on presence of motif neoepitopes and TMB status, with motif present and TMB-H demonstrating improved long-term outcomes as compared to motif absent and/or TMB-L (HR 0.21, p=0.002) (FIG. 18). This suggests that patients with favorable neoepitopes and expression of PD-L1 or TMB-H may be dependent on high levels of the immune checkpoint to prevent immune surveillance and drive tumorigenesis. Although sample size is limited, those with B44 motif matched neoepitopes and high PD-L1 expression appear to disproportionately constitute the long-term survivors.

### DISCUSSION

Over the last two decades, our understanding of cancer immunoediting has evolved and the process is currently recognized as a spectrum of tumor elimination, equilibrium, and escape.(48) However, the dynamic interplay between immunosurveillance and tumor resistance makes the concept difficult to study in humans. Temporal occurrence and extent of tumor cell elimination and escape is not well characterized, and is heterogeneous both between and within different tumor types.(49) As this process has implications in ICB, gleaning a better understanding of immunoediting will aid in development of individualized therapies. In this manuscript, we evaluated mutational landscapes in NSCLC and melanoma tumors for evidence of immunoediting in the context of HLA type and motif matched neoepitopes. Additionally, we investigated for signatures of immune escape in association with favorable neoepitopes. Our previous work uncovered improved survival with ICB in NSCLC and melanoma patients harboring HLA-B44 motif-matched neoepitopes, suggesting these mutations are likely immunogenic. Therefore, we limited our analysis to the HLA-B44 supertype.

We found modest evidence for immunoediting at the DNA level in LUAD and LUSC tumors, in that samples with B44 were less likely to harbor a mutational landscape that enriched for radical negative substitutions than non-B44 tumors. However, this pattern was not observed in SKCM, leading us to postulate that some tumors employ escape mechanisms that bypass DNA editing without depletion of neoepitopes. Upon investigation of neoepitope transcription, we found that motif neoepitopes had decreased expression compared to non-motif neoepitopes in SKCM, which may result in decreased presentation of favorable neoantigens. This process has been observed in tumors while on ICB, and more recently, in a study of glioma patients that had reduced expression of predicted neoantigens with strong binding affinities.(50,51) Similarly, alterations in APM, could restrict the repertoire of neoantigens presented to immune effector cells, putatively leading to immune escape.(52-54)

Immunogenic stimuli modulate the ratio of immune stimulatory and inhibitory cells, subsequently influencing response to immunotherapy. As our focus was on immune escape mechanisms, we were interested in evaluating enrichment of inhibitory cells.(55) However, we were limited to Tregs and M2 macrophages as the xCell software does not infer additional inhibitory cell types such as myeloid derived suppressor cells. We found enrichment of both M2 macrophages and Tregs in LUSC tumors with motif neoepitopes, with M2 macrophages maintaining significance when adjusted for multiple hypothesis testing. Upon assessment of cytokine expression, increased levels of cytokines associated with recruitment of Tregs and polarization of M2 macrophages were observed in LUSC. It is plausible that cytokine signals released by tumor cells in LUSC patients harboring B44 motif-matched neoepitopes could lead to compensatory enrichment of inhibitory cells and subsequent immune escape, however this relationship will require further investigation. Moreover, it was recently shown that Tregs with neoantigen reactivity are frequently found in metastatic tumors, suggesting that these cells act in a tumor antigen-selective manner resulting in activation and clonal expansion in the TME.(56)

With the advent of ICB, understanding the relationship between presence of immunogenic neoantigens and expression of immune checkpoints is important for optimal selection of patients for specific immunotherapies. Here we show that tumors harboring motif neoepitopes have greater expression of certain immune checkpoints that have been recognized to play a role in immune escape.(57) Induction of immune checkpoints putatively eliminates a survival advantage from immune editing and neoepitope depletion at the DNA level. However, after treatment with ICB, neoantigens may be unmasked and subsequently subject to immunoediting, as was shown in an important study where NSCLC tumors lost somatic mutations that putatively produced neoantigens following treatment with anti-PD-1/CTLA-4.(58)

It is probable that tumors with favorable neoepitopes that have undergone immune escape by induction of PD-L1 are particularly dependent on this mechanism to avoid destruction, with ICB treatment exposing tumor cells for destruction. Given that patient with B44 motif-matched neoepitopes were shown to have improved outcomes with ICB in prior studies, and that higher levels of PD-L1 by IHC predicts for better response, we hypothesized that patients who have both are more likely to be long-term survivors. We were interested in investigating this hypothesis in NSCLC given our prior data and experience with immunotherapy. In the NSCLC cohort, although sample size was limited, results showed longer OS and PFS in those with both favorable neoepitopes and high expression of PD-L1. Patients that lack favorable neoepitopes showed limited response, regardless of PD-L1 expression. Those with favorable neoepitopes and low PD-L1 expression also demonstrated inferior response, as these tumors ostensibly rely on mechanisms other than induction of PD-L1 to evade immune surveillance and therefore are less susceptible to anti-PD-1 therapy.

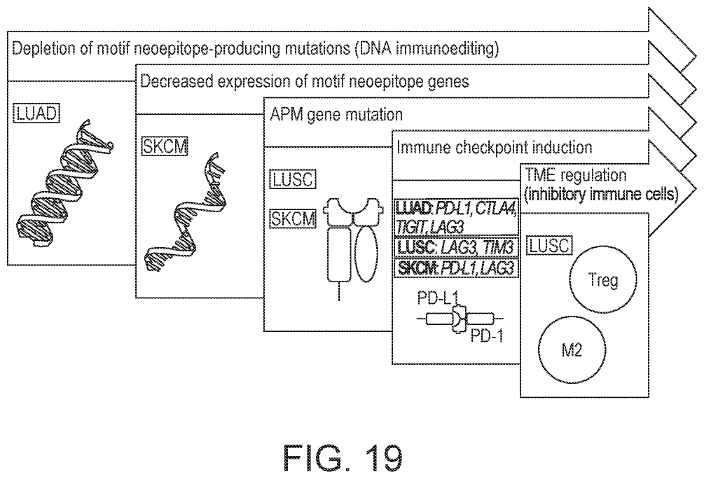

In this study, we systematically evaluated steps involved in neoantigen-directed immune escape by investigating alterations in neoantigen production at the DNA and RNA level, antigen presentation machinery, tumor microenvironment, and immune checkpoints (FIG. 14). We limited our inquiry to pertinent APM genes, immune checkpoint genes, and immune inhibitory cells that have been well established in prior literature.(36,40-43,55) We observed varying degrees of immunoediting and escape among histologies. LUAD demonstrated depletion of motif neoepitope-producing mutations and greatest extent of immune checkpoint induction. LUSC showed mutations in APM genes, enrichment of immune inhibitory cells, and induction of immune checkpoints. SKCM exhibited decreased RNA expression of genes harboring motif neoepitopes, APM gene mutations, and increased expression of certain immune checkpoint genes. These findings are consistent with previous studies demonstrating neoantigen depletion and differences in APM gene mutations and inhibitory TME in NSCLC,(59,60) as well as downregulation of APM in metastatic melanoma.(61) The diverse evasion mechanisms observed by tumor type in this study, occurring at different levels of the anti-tumor immune response, are summarized in FIG. 19.

The discordant immune evasion strategies observed in this study likely reflect the underlying complexities and differences in tumor biology, including but not limited to genetic, epigenetic, and metabolic features of the various tumor types.(62-66) The resulting immunogenomic characteristics likely influence the degree to which different tumor types exploit immune escape mechanisms. Moreover, the diverse evasion strategies can be inferred from heterogenous immunotherapy responses seen among different histologies. Given this complexity in anti-tumor immune response, it is not surprising that we did not identify a universal mechanism of immune escape. The etiology of disparate evasion mechanisms we observed remains unclear and will require future investigation. While trends are observed based on type of cancer, it is important to recognize biological differences within each histology, patient, and individual tumor for successful implementation of precision medicine.(49)

Limitations include the small sample size for survival analysis which was constrained by availability and quality of samples. Availability of data was restricted to samples with adequate sequencing in addition to PD-L1 IHC. This limited comparison of subgroups, and required grouping of samples based on presence of motif neoepitopes and PD-L1 status. Although we were able to access additional cohorts, publicly available data derived from long-term follow up of NSCLC patients treated with single-agent ICB is lacking. Yet, our results suggest that presence of both favorable neoepitopes and PD-L1 expression are important for long-term response to ICB. Although TOGA provides comprehensive multi-omics for many tumor types, there are confines to using the dataset. Samples are largely derived from untreated, early-stage tumors, with the exception of regional metastases in cutaneous melanoma. Additionally, spatial and temporal aspects of tumor heterogeneity are not captured in this dataset. Furthermore, neoantigen prediction remains a nascent field without a gold standard algorithm. A global consortium has formed to address this issue, and a recent study published by the group demonstrated large differences and poor overlap in neoantigen prediction between multiple pipelines.(67) Considering these findings and absence of proteomic and T-cell reactivity data, it is difficult to discern if the predicted neoepitopes in this study truly produce immunogenic neoantigens capable of driving an immune response, although immunogenicity is inferred based on survival analysis. Similarly, cell infiltrate and immune checkpoints are based on gene expression data and lack broad proteomic information or IHC for more definitive characterization.

Although neoantigen-directed immune escape has been previously observed,(60) the method used to define favorable neoepitopes in this study, based on structural features of the HLA-B44 binding pocket, is unique. Moreover, we have previously shown clinical efficacy of ICB in tumors with B44 motif-matched neoepitopes in NSCLC and melanoma, further substantiating our method of determining clinically relevant and likely immunogenic neoepitopes.(14) The results of this study suggest that long-term survivors on anti-PD-1 therapy additionally rely on expression of PD-L1, which is putatively a major immune escape mechanism in tumors with favorable neoepitopes. However, there remain poor responders to ICB in the setting of both favorable neoepitopes and PD-L1 expression, indicating that tumors depend on diverse mechanisms for immune escape, some of which are investigated in this study. These diverse evasion mechanisms will require additional investigation in vivo and can shed light on future efforts to customize immunotherapy.

### REFERENCES

1. Garon E B, et al. N Engl J Med [Internet]. 2015; 372:2018-28.
2. Garon E B, et al. J Clin Oncol [Internet]. 2019; 37:2518-27.
3. Havel J J, et al. Nat Rev Cancer [Internet]. Springer US; 2019; 19:133-50.
4. Le D T, et al. PD-1 N Engl J Med [Internet]. 2015; 372:2509-20.
5. Bonneville R, et al. JCO Precis Oncol. 2017; 1-15.
6. Marabelle A, et al. KEYNOTE-158. Ann Oncol [Internet]. Elsevier Masson SAS; 2019; 30:v477-8.
7. Doherty P., Zinkernagel R. Lancet [Internet]. 1975; 305:1406-9.
8. Chen D S, Mellman INature [Internet]. 2017; 541:321-30.
9. Sidney J, et al. BMC Immunol [Internet]. 2008; 9:1.
10. DiBrino M, et al. Biochemistry [Internet]. 1995; 34:10130-8.
11. Fiorillo M T, Paladini F, Tedeschi V, Sorrentino R. Front Immunol. 2017; 8:1-5.
12. Chowell D, et al. Science (80-) [Internet]. 2018; 359:582-7.
13. Negrao M V et al. J Thorac Oncol [Internet]. Elsevier Inc; 2019; 14:1021-31.
14. Cummings A L, et al. Nat Cancer [Internet]. 2020; Available from: doi.org/10.1038/s43018-020-00140-1
15. Schumacher T N, Scheper W, Kvistborg P. Annu Rev Immunol [Internet]. 2019; 37:173-200.
16. Schreiber R D, Old L J, Smyth M J. Science (80-) [Internet]. 2011; 331:1565-70.
17. Dupage M, Mazumdar C, Schmidt L M, Cheung A F, Jacks T. Nature [Internet]. Nature Publishing Group; 2012; 482:405-9.
18. Matsushita H, et al. Nature [Internet]. 2012; 482:400-4.
19. Dunn G P, Bruce A T, Ikeda H, Old L J, Schreiber R D. Nat Immunol [Internet]. Elsevier; 2002; 3:991-8.
20. Verdegaal E M E, et al. Nature [Internet]. 2016; 536:91-5.
21. Marty R, et al. MHC-I Cell [Internet]. Elsevier; 2017; 171:1272-1283.e15.
22. Castro A, et al. Nat Commun [Internet]. Springer US; 2020; 11:4128.
23. Rooney M S, Shukla S A, Wu C J, Getz G, Hacohen N. Cell [Internet]. Elsevier Inc.; 2015; 160:48-61.
24. Castro A, Ozturk K, Pyke R M, Xian S, Zanetti M, Carter H. BMC Med Genomics [Internet]. 2019; 12:107.
25. McGranahan N, et al. Cell [Internet]. 2017; 171:1259-1271.e11.
26. Gajewski T F, Schreiber H, Fu Y X. Nat Immunol. 2013; 14:1014-22.
27. Szolek A, Schubert B, Mohr C, Sturm M, Feldhahn M, Kohlbacher O. Bioinformatics [Internet]. 2014; 30:3310-6.
28. Cingolani P, et al. Fly (Austin) [Internet]. 2012; 6:80-92.
29. Zerbino D R, et al. Ensembl 2018. Nucleic Acids Res. 2018; 46:D754-61.
30. Hundal J, et al. Genome Med [Internet]. Genome Medicine; 2016; 8:11.
31. Vita R, Mahajan S, et al. The Immune Epitope Database (IEDB): 2018 update. Nucleic Acids Res [Internet]. Oxford University Press; 2019; 47:D339-43.
32. Grimsley G R, Scholtz J M, Pace C N. Protein Sci [Internet]. 2008; 18:NA-NA.
33. Sette A, Sidney J. Immunogenetics [Internet]. 1999; 50:201-12.
34. Mounir M, et al. New functionalities in the TCGAbiolinks package for the study and integration of cancer data from GDC and GTEx. Wang E, editor. PLOS Comput Biol [Internet]. 2019; 15:e1006701.
35. Colaprico A, et al. Nucleic Acids Res. 2016; 44:e71.
36. Leone P, Shin E-C, Perosa F, Vacca A, Dammacco F, Racanelli V. JNCI J Natl Cancer Inst [Internet]. 2013; 105:1172-87.
37. Aran D, Hu Z, Butte A J. Genome Biol [Internet]. Genome Biology; 2017; 18:220.
38. Stockis J, Roychoudhuri R, Halim T Y F. Immunology. 2019; 157:219-31.
39. Wang L, Zhang S, Wu H, Rong X, Guo J. J Leukoc Biol [Internet]. 2019; 106:345-58.
40. Waldman A D, Fritz J M, Lenardo M J. Nat Rev Immunol [Internet]. Springer US; 2020; 20:651-68.
41. Qin S, Xu L, Yi M, Yu S, Wu K, Luo S. Mol Cancer [Internet]. Molecular Cancer; 2019; 18:155.
42. Marin-Acevedo J A, Dholaria B, Soyano A E, Knutson K L, Chumsri S, Lou Y. J Hematol Oncol [Internet]. Journal of Hematology & Oncology; 2018; 11:39.
43. Donini C, D'Ambrosio L, Grignani G, Aglietta M, Sangiolo D. J Thorac Dis [Internet]. 2018; 10:S1581-601.
44. Risso D, Schwartz K, Sherlock G, Dudoit S. G C-Content Normalization for RNA-Seq Data. 2011;
45. Li J, et al. Cancer Res. 2017; 77:e51-4.
46. Rizvi N A, et al. Science (80-). 2015;
47. Anagnostou V, et al. Nat Cancer [Internet]. Springer US; 2020; 1:99-111.
48. Mittal D, Gubin M M, Schreiber R D, Smyth M J. Curr Opin Immunol [Internet]. 2014; 27:16-25.
49. Beatty G L, Gladney W L. Clin Cancer Res [Internet]. 2015; 21:687-92.
50. Nejo T, et al. Cancer Immunol Res [Internet]. 2019; 7:1148-61.
51. Riaz N, Morris L, Havel J J, Makarov V, Desrichard A, Chan T A. Int Immunol. 2016; 28:411-9.
52. Ling A, et al. Oncoimmunology [Internet]. 2017; 6:e1356143.
53. Pereira C, et al. Clin Cancer Res [Internet]. 2017; 23:3203-13.
54. del Campo A B, et al. Int J Cancer [Internet]. 2014; 134:102-13.
55. Galli F, Aguilera J V, Palermo B, Markovic S N, Nisticò P, Signore A. J Exp Clin Cancer Res [Internet]. Journal of Experimental & Clinical Cancer Research; 2020; 39:89.
56. Ahmadzadeh M, et al. Sci Immunol [Internet]. 2019; 4:eaao4310.
57. Wherry E J, Kurachi M. Nat Rev Immunol [Internet]. Nature Publishing Group; 2015; 15:486-99.
58. Anagnostou V, et al. Cancer Discov. 2017; 7:264-76.
59. Faruki H, Mayhew G M, Serody J S, Hayes D N, Perou C M, Lai-Goldman M. J Thorac Oncol [Internet]. Elsevier Inc; 2017; 12:943-53.
60. Rosenthal R, et al. Nature [Internet]. 2019; 567:479-85.
61. Kageshita T, Hirai S, Ono T, Hicklin D J, Ferrone S. Am J Pathol [Internet]. American Society for Investigative Pathology; 1999; 154:745-54.
62. O'Donnell J S, Teng M W L, Smyth M J. Nat Rev Clin Oncol [Internet]. Springer US; 2019; 16:151-67.
63. Campbell P J, et al. Naturek [Internet]. 2020; 578:82-93.
64. Charoentong P, et al. Cell Rep [Internet]. ElsevierCompany.; 2017; 18:248-62.
65. Cristescu R, et al. Science (80-) [Internet]. 2018; 362:eaar3593.
66. Thorsson V, et al. Immunity [Internet]. 2018; 48:812-830.e14.
67. Wells D K, et al. Cell. 2020; 183:818-834.e13.

### Example 3: Exposure of Autologous DCs to Motif Neoepitopes for Enhanced Systemic Motif Neoantigen-Specific T Cell Activation and Expansion

This Example describes methods to evaluate the potential of autologous dendritic cells (DCs) exposed to motif neoepitopes in promoting systemic motif neoepitope-specific T cell activation. Patient biospecimens collected from an ongoing phase I trial of intratumural (IT) administration of CCL21-DC combined with pembrolizumab in NSCLC will be used to evaluate whether exposing autologous DCs to motif neoepitopes provides as an innovative approach for personalized medicine to amplify tumor-specific T cell activation. This strategy supports clinical interventions in which autologous DCs are exposed to motif neopitopes and administrated back to the patient to enhance immune destruction of the tumor. We hypothesize that exposing DCs to motif neoepitopes, particularly at an optimal dose and schedule, will potentiate activation and expansion of motif neoepitope-specific T cells.

Exposing Autologous DCs to Motif Neoepitopes for Enhanced Systemic Motif Neoantigen-Specific T Cell Activation and Expansion

The current phase I study evaluates the safety of intratumoral CCL21-DC in combination with IV pembrolizumab in patients with stage IV NSCLC without epidermal growth factor receptor (EGFR) or anaplastic lymphoma kinase (ALK) alterations, who have progressed on prior PD-1/PD-L1 inhibitors with or without chemotherapy; or patients with EGFR and/or ALK alterations with disease progression on tyrosine kinase inhibitor (TKI) therapy without prior anti-PD-1/PD-L1 therapy. This is a single institution, non-randomized, dose-escalation, multi-cohort trial followed by an expansion cohort at the maximum tolerated dose (MTD) established during dose escalation. Nine patients will be evaluated in a dose escalation cohort (1×10⁷, 3×10⁷ cells/injection). To date, three patients were enrolled in the initial cohort without a dose limiting toxicity (DLT). Three additional patients have completed the DLT period at the second dose level without a DLT. An additional 15 patients will be enrolled. Patients receive three IT injections of autologous CCL21-DC (days 0, 21, 42) together with IV pembrolizumab. Pembrolizumab continues every 3 weeks for up to 1 year. This trial affords the opportunity to collect a baseline biopsy (Day 0) and serial tumor biopsies following each IT injection (Days 21, 42), together with temporal blood samples following treatments (Days 0, 11, 21, 42, 63, 126, 189, 252, 315), along with untransduced autologous DCs. We plan to use this trial as a platform to evaluate the potential of autologous DCs exposed to motif neoepitopes in promoting systemic motif neoepitope-specific T cell activation, which provides a novel strategy to enhance the efficacy of current immunotherapies.

Patients with HLA-B44 supertype in the presence or absence of motif neoepitopes will be included in these studies, and PBMCs collected at various time points (Days 0, 11, 21, 42, 63, 126, 189, 252, 315) will be utilized for co-culturing with autologous DCs exposed to motif neoepitopes from the same patient. All identified motif neoepitopes will be evaluated, and the top 5 computationally identified putative neoepitopes of individual patients will be included for comparison. Briefly, CD3+ T cells will be isolated from PBMCs utilizing human CD3+ T cell enrichment magnetic beads (Stemcell Technologies). DCs (2×10⁵) will be incubated with their respective motif or non-motif neoepitopes (1 ng/ml) at 37° C. for 2 hours, irradiated at 6,000 rad, and then co-incubated with 2×10⁶ autologous T cells supplemented with IL-15 (10 ng/ml) and IL-2 (10 IU/ml) for 12 days. T cells will be restimulated with the corresponding peptides, followed by intracellular cytokine or tetramer staining and flow cytometry. The kinetics and magnitude of tumor-specific systemic T cell activation will be correlated with the presence or absence of motif neoepitopes. These studies can be used to identify the most potent motif neoepitopes to drive systemic tumor-specific T cell activation and the best time point for detection.

Statistical Analysis

We will utilize mixed effects analysis of variance models to evaluate the effects of experimental factors on the outcomes of the set of in vitro experiments for each subject/neoepitope. These models will evaluate multiple outcome measures (ex. T-cell activation, cytokines) for each experiment. The main effects will be sampling time, motif neoepitopes (presence or absence), and the time by motif enoepitope interaction effect. A random effect for subject will be included to account for repeated observations over time within subjects.

Identifying Optimal Conditions of Exposing Autologous DCs to Motif Neoepitopes as a Therapeutic Strategy to Promote Systemic Motif Neoantigen-Specific T Cell Activation

To harness the antigen presenting function of DCs for potential clinical translation, we plan to identify the optimal conditions of exposing autologous DCs with motif neoepitopes in promoting neoepitope-specific T cell activation by in vitro assays. Samples from patients possessing motif neoepitopes that demonstrate the most effective peripheral neoepitope-specific T cell activations from the above studies will be subjected to the following. Similar DC/T cell co-cultures will be established as detailed above, and we will test the following parameters: 1) DC:T cell ratio (DC:T=1:3, 1:5, 1:10 with T cells fixed at 2×10⁶) and 2) incubation time (5, 7, 9 or 12 days of co-culture). We will also conduct these experiments with or without PD-1 inhibition (pembrolizumab at 2 μg/ml) to evaluate whether PD-1 blockade can synergize with DC to promote neoepitope-specific T cell activation in this setting. T cell enumeration and viability will be determined by trypan blue staining and cell counting at the time of harvesting. T cell activation will be assessed by restimulation with DCs preloaded with the same peptide for 16 h (with protein transport inhibitor Brefordin A during the last 4 h of incubation), followed by intracellular cytokine staining of IFN-γ and TNF-α. Tetramer staining will also be performed in parallel to confirm that cytokine-secreting T cells are motif neoepitope-specific. These studies will identify the optimal conditions for neoepitope-exposed DCs to drive neoepitope-specific T cell activation and expansion.

We will utilize analysis of variance models to evaluate the effects of experimental factors on the outcomes of the set of in vitro experiments for each subject/neoepitope. These models will evaluate multiple outcome measures (e.g., T-cell activation, cytokines) for each experiment. Main effects will include motif neoepitopes (presence or absence), T cell ratios, incubation time and presence or absence of PD-1 inhibition. We will also include terms for interactions between the main effects. Particular interest will be the PD-1 by motif neoepitope interaction effect, which would suggest that the specific motifs confer differential response to PD-1 inhibition.

Similar studies can also be performed with biospeciemens collected from surgical patients. Tumor cell lines derived from the surgical samples could be utilized as autologous targets in cytotoxicity assays of DC-activated motif neoantigen-specific T cells to assess the tumor killing capacity of these T cells.

In summary, we propose to comprehensively evaluate the immunologically/clinically relevant effects of motif neoepitopes. These studies include correlating features of the TME with motif neoepitopes in early and advanced stage NSCLC patients, using slide review, computational analyses based on gene expression, MIF and scRNA. In advanced stage patients, we will also correlate these features with clinical outcome among patients who received single agent PD-1 inhibition. Finally, we will use our unique repository of autologous DCs and PBMCs from well characterized patients to evaluate the potential for exposing DCs to motif neoepitopes to enhance the cytotoxic potential of motif neoepitope-specific T cells. These studies will greatly enhance our understanding of neoepitope induced changes in the tumor microenvironment, refine the use of motif neoepitopes as a biomarker for efficacy of PD-1 inhibition in NSCLC, and lay the foundation for future clinical investigation of motif neoepitope-exposed autologous DC with or without PD-I inhibition for NSCLC.

### Example 4: RNA Vaccines

The principles described herein can be applied to other methods of delivery of the neoeptiopes for cancer immunotherapy. An alternative to using dendritic cells for delivery would be to construct an RNA vaccine. In one example, one could construct an RNA vaccine for high risk patients for EGFR L858R, a peptide resultant from a potential motif neoepitope. Such a vaccine would be desirable for use with patients at risk for EGFR mutations. Other constructs for nucleic acid delivery are contemplated.

Mutations in the epidermal growth factor receptor (EGFR) gene are common among lung cancer patients who were never smokers. Although immunotherapies have revolutionized the management of lung cancer, this population has experienced little benefit from immunotherapy. As shown above, among patients with charged HLA-B supertype alleles (B44 and B27), outcomes with immunotherapy are better in patients with an oppositely charged mutant amino acid (with a change in electrostatic charge from wildtype) in the second position of a nonamer. Among patients with HLA-B27 alleles, the common L858R gene in EGFR is such a mutation. Responses to immunotherapy are rare, but seen, in EGFR mutant NSCLC. One can thus evaluate the immunologic changes seen in EGFR L858R mutant patients with HLA-B27 alleles, and assesses whether exposing dendritic cells to the resultant peptides can generate a personalized immunotherapy approach. Likewise, one can construct a nucleic acid construct, such as an RNA vaccine, to deliver the peptides or to direct the expression of the peptides.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of treating cancer in a subject, the method comprising:
(a) obtaining antigen presenting cells (APCs);
(b) pulsing the APCs with a neoepitope associated with the subject's cancer; and
(c) administering the pulsed APCs to the subject;
wherein the neoepitope is a nonamer comprising a radical substitution in the second position.

2. The method of claim 1, wherein the APCs are dendritic cells.

3. The method of claim 1, wherein the APCs are autologous.

4. The method of claim 1, wherein the neoepitope associated with the subject's cancer comprises an amino acid sequence encoded by a nucleic acid sequence obtained by sequencing a biological sample obtained from the subject.

5. A method of identifying a cancer as responsive to immune checkpoint blockade (ICB), the method comprising:
(a) obtaining a biological sample of the cancer from a subject;
(b) sequencing nucleic acid from the biological sample; and
(c) identifying the cancer as ICB responsive when the sequencing detects a nucleic acid encoding a neoepitope, wherein the neoepitope is a nonamer comprising a radical substitution in the second position.

6. The method of claim 1, wherein the subject expresses the human leukocyte antigen (HLA) supertype B44 and/or B27.

7. The method of claim 1, wherein the subject expresses the HLA supertype B44, and wherein the radical substitution consists of a negatively charged amino acid.

8. The method of claim 1, wherein the subject expresses the HLA supertype B27, and wherein the radical substitution consists of a positively charged amino acid.

9. The method of claim 7, wherein the negatively charged amino acid is glutamic acid or aspartic acid.

10. The method of claim 8, wherein the negatively charged amino acid is glutamic acid.

11. The method of claim 8, wherein the positively charged amino acid is histidine, lysine, or arginine.

12. The method of claim 1, wherein the cancer is non-small cell lung cancer (NSCLC).

13. The method of claim 1, wherein the cancer is melanoma.

14. The method of claim 4, wherein the biological sample is a tumor specimen.

15. The method of claim 4, wherein the biological sample comprises circulating tumor DNA (ctDNA).

16. The method of claim 1, further comprising administering to the subject a PD-1 inhibitor.
